(19) United States
(12) Patent Application Publication
(10) Pub. No.: US 2025/0123287 A1
(43) Pub. Date: Apr. 17, 2025

(54) COMPOSITIONS AND METHODS FOR CHARACTERIZING MULTISPECIFIC ANTIGEN BINDING MOLECULES FROM SINGLE CELLS

(71) Applicant: 10x Genomics, Inc., Pleasanton, CA (US)

(72) Inventor: Wyatt James McDonnell, Concord, CA (US)

(21) Appl. No.: 18/987,399

(22) Filed: Dec. 19, 2024

Related U.S. Application Data
(63) Continuation of application No. PCT/US2023/068886, filed on Jun. 22, 2023.
(60) Provisional application No. 63/354,940, filed on Jun. 23, 2022.

(51) Int. Cl. G01N 33/68; G01N 33/569

(52) U.S. Cl. CPC G01N 33/6845; G01N 33/56983; G01N 2333/08; G01N 2333/135; G01N 2458/10

(57) ABSTRACT

The present disclosure relates generally to compositions, methods, and systems for the characterization of antigen-binding molecules, e.g., multispecific T cell receptors, B cell receptors, antibodies, and antigen-binding fragments thereof, using single-cell immune profiling methodologies. The compositions, methods and systems described herein permit rapid, high-throughput identification and characterization of multispecific antigen binding molecules having desired properties.

### CROSS-REFERENCE TO RELATED APPLICATIONS

This application is a continuation of PCT/US2023/068886, filed Jun. 22, 2023, which claims benefit of and priority to U.S. Provisional Application Ser. No. 63/354,940 filed Jun. 23, 2022, the entire contents of which are incorporated herein by reference for all purposes.

### REFERENCE TO AN ELECTRONIC SEQUENCE LISTING

The contents of the electronic sequence listing (900299.425C1_SEQUENCE_LISTING.xml; Size: 9,758 bytes; and Date of Creation: Aug. 25, 2023) is herein incorporated by reference in its entirety.

### BACKGROUND

Antigen binding molecules (ABMs) that bind to antigens of interest can be developed as new immunotherapeutic agents. Most ABMs for use in therapy are monospecific, e.g., are specific for a single antigen. However, multispecific ABMs are gaining heightened interest, as they offer a number of advantages over monospecific ABMs. For instance, multispecific ABMs may be specific for different molecules on the surface of single cell that are involved in a certain type of signaling, e.g, inflammatory or cell growth, pathway. As a result, multispecific ABMs having specificity for the different molecules involved in that certain signaling pathway may have an augmented therapeutic effect on an undesirable actitivity, e.g., inflammation or tumorigenesis, mediated by the cell. Multispecific ABMs may also be capable of binding to epitopes of more than one pathogenic agent, e.g., viral agent, or of binding to multiple epitopes of a single pathogenic agent, increasing their usefulness as a therapeutic. Multispecific ABMs are advantageous not only due to their enhanced clinical effectiveness, but also because the costs of developing and manufacturing a single multispecific ABM are less than that to develop and manufacture several monospecific ABMs that separately bind the set of antigens bound by the single multispecific ABM.

There is a need for high-throughput methods and reagents that reliably and rapidly characterize and identify multispecific immunotherapeutic molecules, e.g., multispecific ABMs.

### SUMMARY

Provided herein are, inter alia, methods, compositions and systems useful for characterization of (ABMs), such as T cell receptors (TCRs), B cell receptors (BCRs), antibodies (Abs) or antigen-binding fragments thereof. Characterization of ABMs having desirable properties, e.g., that are multispecific or have the ability to recognize and bind to more than one target antigen, can be useful in the development of new immunotherapies to treat cancers, autoimmune and/or infectious disease. Also provided in some embodiments of the disclosure are kits for the discovery and/or characterization of these ABMs, e.g., TCRs, BCRs, Abs or antigen-binding fragments thereof that may be multispecific.

In an aspect, the description provides for a first method for characterizing an ABM. The method includes a step of partitioning a reaction mixture, or a portion thereof, into a plurality of partitions. The reaction mixture contains a plurality of cells expressing ABMs and a plurality of target reagents. The plurality of target reagents includes a first target reagent. The first target reagent includes: (i) a first target antigen, (ii) a second target antigen and (iii) a first reporter oligonucleotide. The step of partitioning provides a partition that contains: (i) a cell expressing an ABM that had bound to the first target reagent, (ii) the first target reagent and (iii) a plurality of nucleic acid barcode molecules that have a partition-specific barcode sequence. Barcoded nucleic acid molecules are generated. The barcoded nucleic acid molecules include: (i) a first barcoded nucleic acid molecule having a first nucleic acid sequence encoding at least a portion of the ABM expressed by the cell or a reverse complement thereof and the partition-specific barcode sequence or a reverse complement thereof, and (ii) a second barcoded nucleic acid molecule including a sequence of the first reporter oligonucleotide or a reverse complement thereof and the partition-specific barcode sequence or reverse complement thereof.

Some embodiments of the first method further include characterizing the ABM expressed by the cell as a candidate ABM having specificity for the first and the second target antigen, based on the generated second barcoded nucleic acid molecule.

In some of any of the embodiments of the first method, the first reporter oligonucleotide includes a first reporter barcode sequence. In some embodiments, the first reporter barcode sequence identifies the first target reagent to which it is coupled.

In some embodiments of the first method, the first target reagent further includes: (iv) an additional reporter oligonucleotide, wherein the first target antigen is coupled to the first reporter oligonucleotide and the second target antigen is coupled to the additional reporter oligonucleotide. In some embodiments, the first reporter oligonucleotide includes a first reporter barcode sequence, and the additional reporter oligonucleotide includes an additional reporter barcode sequence. In some embodiments, the first reporter barcode sequence identifies the first target antigen and the additional reporter barcode sequence identifies the second target antigen. In some embodiments, the barcoded nucleic acid molecules generated in the method further include an additional barcoded nucleic acid molecule, wherein the additional barcoded nucleic acid molecule includes a sequence of the additional reporter oligonucleotide or a reverse complement thereof and the partition-specific barcode sequence or reverse complement thereof.

In some of any of the embodiments of the first method, the plurality of target reagents further includes: a second target reagent and a third target reagent, wherein the second target reagent includes the first target antigen and a second reporter oligonucleotide; and the third target reagent includes the second target antigen and a third reporter oligonucleotide. In some embodiments, the second reporter oligonucleotide includes a second reporter barcode sequence, and the third reporter oligonucleotide comprises a third reporter barcode sequence. In some embodiments, the second reporter barcode sequence identifies the second target reagent and the third reporter barcode sequence identifies the third target reagent.

In some embodiments of the first method in which the plurality of target reagents further include the second target reagent and the third target reagent, the cell (that had bound to the first target reagent) had further bound to both the second and the third target reagent, and the barcoded nucleic acid molecules generated further include third and fourth barcoded nucleic acid molecules, wherein the third barcoded nucleic acid molecule includes a sequence of the second reporter oligonucleotide or a reverse complement thereof and the partition-specific barcode sequence or reverse complement thereof, the fourth barcoded nucleic molecule includes a sequence of the third reporter oligonucleotide or reverse complement thereof and the partition-specific barcode sequence or reverse complement thereof. Some embodiments further include characterizing the candidate ABM as having specificity for both the first and the second target antigen based on the generated third and fourth barcoded nucleic acid molecules.

In some embodiments of the first method in which the plurality of target reagents further include the second target reagent and the third target reagent, the cell (that had bound to the first target reagent) had further bound to the second, but not the third, target reagent, and the barcoded nucleic acid molecules generated further include a third barcoded nucleic acid molecule, wherein the third barcoded nucleic acid molecule includes a sequence of the second reporter oligonucleotide or a reverse complement thereof and the partition-specific barcode sequence or reverse complement thereof. Some embodiments further include characterizing the candidate ABM as having specificity for the first, but not the second, target antigen based on the generated third barcoded nucleic acid molecule.

In some embodiments of the first method in which the plurality of target reagents further include: the second target reagent and the third target reagent, the cell (that had bound to the first target reagent) had further bound to the third, but not the second, target reagent, and the barcoded nucleic acid molecules generated further include a third barcoded nucleic acid molecule, wherein the third barcoded nucleic acid molecule includes a sequence of the third reporter oligonucleotide or a reverse complement thereof and the partition-specific barcode sequence or reverse complement thereof. Some embodiments further include characterizing the candidate ABM as having specificity for the second, but not the first, target antigen based on the generated third barcoded nucleic acid molecule.

In any of the embodiments of the first method, the first reagent further includes a first detectable label. In some embodiments, the first detectable label is fluorescent or magnetic. In some embodiments, the first detectable label is a fluorescent molecule. In some embodiments, prior to the partitioning, cells of the plurality of cells having the detectable label are sorted.

In some embodiments of the first method in which the first target reagent further includes a fluorescent molecule as a first detectable label, the first target reagent further includes a second detectable label, wherein the first fluorescent molecule is coupled to the first target antigen and the second fluorescent molecule is coupled to the second target antigen, wherein the first and the second fluorescent molecules emit first and second detectable signals. In some embodiments, prior to the partitioning, cells of the plurality of cells having the first and the second fluorescent molecule, are sorted.

In some of any of the embodiments of the first method: (i) a first nucleic acid barcode molecule of the plurality of nucleic acid barcode molecules further includes a capture sequence configured to couple to (a) an mRNA or DNA analyte or (b) non-templated nucleotides appended to a cDNA reverse transcribed from an mRNA analyte; and (ii) a second nucleic acid barcode molecule of the plurality of nucleic acid barcode molecules further includes a capture sequence configured to couple to a capture handle sequence further included in the first reporter oligonucleotide.

In another aspect, the description provides for a further, second, method for characterizing an ABM. The method includes a step of partitioning a reaction mixture, or a portion of thereof, into a plurality of partitions. The reaction mixture contains a plurality of cells expressing ABMs and a plurality of target reagents. The plurality of target reagents includes a first target reagent. The first target reagent includes: (i) a first target antigen, (ii) a second target antigen and (ii) a first fluorescent molecule. The step of partitioning provides a partition that contains: (i) a cell expressing an ABM that had bound to the first target reagent, (ii) the first target reagent and (iii) a plurality of nucleic acid barcode molecules that have a partition-specific barcode sequence. Barcoded nucleic acid molecules are generated. The barcoded nucleic acid molecules include a first barcoded nucleic acid molecule having a first nucleic acid sequence encoding at least a portion of the ABM expressed by the cell or a reverse complement thereof and the partition-specific barcode sequence or a reverse complement thereof.

Some embodiments of the second method further include characterizing the ABM based on the generated first barcoded nucleic acid molecule.

Some of any of the embodiments of the second method further include, prior to the partitioning, sorting for cells of the plurality of cells that include the first fluorescent molecule.

In some of any of the embodiments of the second method, the first target reagent further includes an additional fluorescent molecule, wherein the first target antigen is coupled to the first fluorescent molecule and the second target antigen is coupled to the additional fluorescent molecule, wherein the first fluorescent molecule emits a first detectable signal and the additional fluorescent molecule emits an additional detectable signal. Some such embodiments further include, prior to the partitioning, sorting for cells of the plurality of cells that include the first and the additional fluorescent molecule.

In some of any of the embodiments of the second method, the plurality of target reagents further includes: a second target reagent and a third target reagent, wherein the second target reagent includes the first target antigen and a second fluorescent molecule; and wherein the third target reagent includes the second target antigen and a third fluorescent molecule. Some embodiments further include, prior to the partitioning, sorting for cells of the plurality of cells that include the first, the second and the third fluorescent molecule.

In any of the embodiments of the second method, a first nucleic acid barcode molecule of the plurality of barcode molecules further includes a capture sequence configured to couple to: (a) an mRNA or DNA analyte or (b) non-templated nucleotides appended to a cDNA reverse transcribed from an mRNA analyte.

In some embodiments of the first or second method in which the first nucleic acid barcode molecule of the plurality of barcode molecules further includes a capture sequence, the capture sequence is configured to couple to (a) the mRNA or DNA analyte; or (b) the non-templated nucleotides appended to the cDNA reverse transcribed from the mRNA analyte. In some embodiments, embodiments in which the capture sequence is configured to couple to the (a) mRNA or cDNA analyte, the capture sequence is configured to couple to the mRNA analyte. In some embodiments, the capture sequence configured to couple to the mRNA analyte includes a polyT, a gene-specific or a random sequence. In some embodiments, embodiments in which the capture sequence is configured to couple to the (b) non-templated nucleotides appended to the cDNA reverse transcribed from the mRNA analyte, the mRNA analyte is reverse transcribed to the cDNA utilizing a primer including a polyT sequence, a gene-specific sequence or a random sequence. In some embodiments, the non-templated nucleotides appended to the cDNA include a cytosine. In some embodiments, the capture sequence configured to couple to the cDNA include a guanine. In some embodiments, coupling of the capture sequence to the non-templated cytosine extends reverse transcription of the cDNA into the first nucleic acid barcode molecule to generate the first barcoded nucleic molecule. In some embodiments, the first nucleic acid barcode molecule further includes a template switch oligonucleotide (TSO).

Some of any of the embodiments of the first or second method further include determining the nucleic acid sequence encoding at least the portion of the ABM from the first barcoded nucleic acid molecule. In some embodiments, the nucleic acid sequence encoding the at least the portion of the ABM includes one or more of a complementarity determining region (CDR), a framework (FWR), a variable heavy chain domain (VH), or a variable light chain domain (VL) of the cell-expressed ABM. In some embodiments, an amino acid sequence of the cell-expressed ABM is determined from the determined nucleic acid sequence.

In some of any of the embodiments of the first or second method, the first or second target antigen includes a pathogen, a cytokine, an autoantigen, a tumor-associated antigen, a nanoparticle, a full-length protein, a fragment of a full-length protein, or an MHC molecule. In some embodiments, the pathogen includes a virus or a bacterial cell. In some embodiments, the target antigen is the MHC molecule and the MHC molecule is complexed with a target antigenic peptide, target lipid or target small molecule.

In some of any of the embodiments of the first or second method, the reaction mixture further includes a control agent. In some embodiments, the control reagent includes a control antigen and a control reporter oligonucleotide. In some embodiments, the control antigen includes a serum albumin peptide, or a peptide to which the plurality of cells are naïve. In some embodiments, the control reporter oligonucleotide includes a control reporter barcode sequence that identifies the control antigen. In some embodiments, the control reagent further includes a detectable label. In some embodiments, the detectable label includes a fluorescent molecule.

In some of any of the embodiments of the first and or second method, the reaction mixture further includes a further target reagent, wherein the further target reagent includes a further target antigen and a further reporter oligonucleotide. In some embodiments, the further reporter oligonucleotide includes a further reporter barcode sequence that identifies the further target antigen.

In some of any of the embodiments of the first or second method, the target reagent further includes a core support, wherein the first and the second target antigens are coupled to the core support.

In some embodiments of the first or second method, embodiments in which the plurality of target reagents further include (a) the second target reagent (including the first target antigen and the second reporter oligonucleotide) and the third target reagent (including the second target antigen and the third reporter oligonucleotide); or (b) the second target reagent (including the first target antigen and a second fluorescent molecule) and the third target reagent (including the second target antigen and the thrid fluorescent molecule), the second and the third target reagent further include a core support, wherein the second target reagent includes the first target antigen coupled to the core support, and the third target reagent includes the second target antigen coupled to the core support. In some embodiments, the first and the second target antigen are biotinylated.

In some of any of the embodiments of the first or second method in which the reaction mixture further includes a control reagent, the control reagent further includes a core support, wherein the control antigen is coupled to the core support. In some embodiments, the control antigenis biotinylated.

In some of any of the embodiments of the first or second method in which the reaction mixture further includes a further reagent, the further reagent further includes a core support, wherein the further target antigen is coupled to the core support. In some embodiments, the further target antigen is biotinuylated.

In some of any of the embodiments of the first or second method in which the first target antigen, the second target antigen, the control antigen or the further target antigen is biotinylated, the core support includes streptavidin, avidin, deglycosylated avidin, traptavidin, tamavidin, xenavidin, or bradavidin.

In some of any of the embodiments of the first or second method, the reaction mixture further includes a core support control, wherein the core support control includes a core support coupled to biotin, wherein the core support includes streptavidin, avidin, deglycosylated avidin, traptavidin, tamavidin, xenavidin, or bradavidin.

In some of any of the embodiments of the first or second method, the plurality of cells are obtained from a subject who has been exposed to, is suspected of having been exposed to, or has been immunized with the target antigen. In some embodiments, the subject is a vertebrate. In some embodiments, the subject is a mammal. In some embodiments, the mammal is a human or a mouse.

In some of any of the embodiments of the first or second method, the plurality of cells include T cells, B cells, or B and T cells.

In some of any of the embodiments of the first or second method, the partition is a droplet, well or microwell.

In a further aspect, the disclosure provides a first composition. The first composition includes a target reagent. The target reagent includes a first target antigen, a second target antigen and a first reporter oligonucleotide.

In some embodiments of the first composition, the first reporter oligonucleotide includes a first reporter barcode sequence. In some embodiments, the first reporter barcode sequence identifies the target reagent.

In some embodiments of the first composition, the target reagent further includes an additional reporter oligonucleotide, wherein the first target antigen is coupled to the first reporter oligonucleotide and the second target antigen is coupled to the additional reporter oligonucleotide. In some embodiments, the first reporter oligonucleotide includes a first reporter barcode sequence and the additional reporter oligonucleotide includes an additional reporter barcode sequence. In some embodiments, the first reporter barcode sequence identifies the first target antigen and the additional reporter barcode sequence identifies the second target antigen.

In some of any of the embodiments of the first composition, the target reagent further includes a detectable label. In some embodiments, the detectable label includes a first fluorescent molecule. In some embodiments, the target reagent further includes an additional fluorescent molecule, wherein the first target antigen is coupled to the first fluorescent molecule and the second target antigen is coupled to the additional fluorescent molecule, wherein the first fluorescent molecule emits a first detectable signal and the additional fluorescent molecule emits a second detectable signal.

Some of any of the embodiments of the first composition further include a second and/or a third target reagent, wherein the second target reagent includes the first target antigen and a second reporter oligonucleotide; and the third target reagent includes the second target antigen and a third reporter oligonucleotide. In some embodiments, the second reporter oligonucleotide includes a second reporter barcode sequence and the third reporter oligonucleotide includes a third reporter barcode sequence. In some embodiments, the second reporter barcode sequence identifies the first target antigen and the third reporter barcode sequence identifies the second target antigen.

In yet a further aspect, the disclosure provides a second composition. The second composition includes a target reagent. The target reagent includes a first target antigen, a second target antigen and a first fluorescent molecule.

In some embodiments of the second composition, the target reagent further includes an additional fluorescent molecule, wherein the first target antigen is coupled to the first fluorescent molecule and the second target antigen is coupled to the additional fluorescent molecule, wherein the first fluorescent molecule emits a first detectable signal and the additional fluorescent molecule emits an additional detectable signal.

Some of any of the embodiments of the second composition further include a second target reagent and/or a third target reagent, wherein the second target reagent includes the first target antigen and a second fluorescent molecule; and the third target reagent includes the second target antigen and a third fluorescent molecule.

Some of any of the embodiments of the first or second composition further include a control reagent. In some embodiments, the control reagent includes a control antigen and a control reporter oligonucleotide. In some embodiments, the control antigen includes a serum albumin peptide, or a peptide to which the plurality of cells are naïve. In some embodiments, embodiments in which the control reagent includes the control antigen and the control reporter oligonucleotide, the control reporter oligonucleotide includes a control reporter barcode sequence that identifies the control antigen. In some embodiments, embodiments in which the control reagent includes the control antigen and the control reporter oligonucleotide, the control reagent further includes a detectable label.

Some of any of the embodiments of the first or second composition further include a further target reagent, wherein the further target reagent includes a further target antigen and a further reporter oligonucleotide. In some embodiments, the further reporter oligonucleotide includes a further reporter barcode sequence that identifies the further target antigen.

In some of any of the embodiments of the first or second composition, the target reagent further includes a core support, wherein the first and the second target antigens are coupled to the core support. In some embodiments, the first and the second target antigen are biotinylated.

In some of any of the embodiments of the first or second composition, embodiments in which the composition includes the second and/or third reagent, the second and/or third reagent further includes a core support, wherein the second target reagent comprises the first target antigen coupled to the core support, and the third target reagent comprises the second target antigen coupled to the core support. In some embodiments, the first and the second target antigen are biotinylated.

In some of any of the embodiments of the first or second composition wherein the composition includes a control reagent, the control reagent further includes a core support, wherein the control antigen is coupled to the core support. In some embodiments, the control antigen is biotinylated.

In some of any of the embodiments of the first or second composition wherein the composition includes a further target reagent, the further target reagent further includes a core support, wherein the further target antigen is coupled to the core support. In some embodiments, the further target antigen is biotinylated. In some of any of the embodiments of the first or second composition wherein the first and the second target antigen, the control antigen, or the further target antigen are biotinylated, the core support includes streptavidin, avidin, deglycosylated avidin, traptavidin, tamavidin, xenavidin, or bradavidin.

Some of any of the embodiments of the first or second composition further include a core support control, wherein the core support control includes a core support coupled to biotin, wherein the core support includes streptavidin, avidin, deglycosylated avidin, traptavidin, tamavidin, xenavidin, or bradavidin.

In some of any of the embodiments of the first or second composition, the first or second target antigen includes a pathogen, a cytokine, an autoantigen, a tumor-associated antigen, a nanoparticle, a full-length protein, a fragment of a full-length protein, or an MHC molecule. In some embodiments, the pathogen includes a virus or a bacterial cell. In some embodiments, the MHC molecule is complexed with a target antigenic peptide, target lipid or target small molecule.

Some of any of the embodiments of the first or second composition further include a cell. In some embodiments, the cell is a B cell or a T cell.

In some of any of the embodiments of the first or second composition, the composition is included in a partition. In some embodiments, the partition includes a well, microwell, or a droplet. In some embodiments in which the composition is included in the partition, the composition further includes a plurality of nucleic acid barcode molecules that include a partition-specific barcode sequence.

Any embodiments of the compositions provided herein, with the exclusion of those including a cell or that are included in a partition, may be in a kit with instructions for use.

In an additional aspect, the disclosure provides a first system for characterizing an ABM. The first system includes a target reagent. The target reagent includes a first target antigen, a second target antigen and a first reporter oligonucleotide.

In some embodiments of the first system, the first reporter oligonucleotide includes a first reporter barcode sequence.

In some embodiments of the first system, the target reagent further includes an additional reporter oligonucleotide, wherein the first target antigen is coupled to the first reporter oligonucleotide and the second target antigen is coupled to the additional reporter oligonucleotide.

In some of any of the embodiments of the first system, the target reagent further includes a detectable label. In some embodiments, the detectable label includes a first fluorescent molecule. In some embodiments, the target reagent further includes a second fluorescent molecule, wherein the first target antigen is coupled to the first fluorescent molecule and the second target antigen is coupled to the second fluorescent molecule, wherein the first fluorescent molecule emits a first detectable signal and the second fluorescent molecule emits a second detectable signal.

Some of any of the embodiments of the first system further include a second and/or third target reagent, wherein the second target reagent comprises the first target antigen and a second reporter oligonucleotide; and the third target reagent comprises the second target antigen and a third reporter oligonucleotide.

In another aspect, the disclosure provides a second system for characterizing an ABM. The second system includes a target reagent. The target reagent includes a first target antigen, a second target antigen and a first fluorescent molecule.

In some embodiments of the second system, the target reagent further includes an additional fluorescent molecule, wherein the first target antigen is coupled to the first fluorescent molecule and the second target antigen is coupled to the additional fluorescent molecule, wherein the first fluorescent molecule emits a first detectable signal and the additional fluorescent molecule emits an additional detectable signal.

Some of any of the embodiments of the second system further include a second target reagent and/or a third target reagent, wherein the second target reagent includes the first target antigen and a second fluorescent molecule; and the third target reagent includes the second target antigen and a third fluorescent molecule.

Some of any of the embodiments of the first or second system further include a control reagent. In some embodiments, the control reagent includes a control antigen and a control reporter oligonucleotide. In some embodiments, the control antigen includes a serum albumin peptide, or a peptide to which the plurality of cells are naïve. In some embodiments in which the first or second system further includes the control reagent, the control reagent further includes a detectable label.

Some of any of the embodiments of the first or second system further include a further target reagent, wherein the further target reagent includes a further target antigen and a further reporter oligonucleotide.

Some of any of the embodiments of the first or second system further include a core support control, wherein the core support control includes a core support coupled to biotin, wherein the core support includes streptavidin, avidin, deglycosylated avidin, traptavidin, tamavidin, xenavidin, or bradavidin.

In some of any of the embodiments of the first or second system, the first or second target antigen includes a pathogen, a cytokine, an autoantigen, a tumor-associated antigen, a nanoparticle, a full-length protein, a fragment of a full-length protein, or an MHC molecule. In some embodiments, the pathogen includes a virus or a bacterial cell. In some embodiments, the MHC molecule is complexed with a target antigenic peptide, target lipid or target small molecule.

Some of any of the embodiments of the first or second system further include a plurality of nucleic acid barcode molecules including a partition-specific barcode sequence and a capture sequence.

Some of any of the embodiments of the first or second system further include a partitioning system for generating a partition. In some embodiments, the partitioning system is a microfluidic device.

Some embodiments of the first or second system, embodiments in which the system includes the plurality of nucleic acid barcode molecules including the partition-specific barcode sequence and the capture sequence, are embodiments that further include reagents for generating a first of a plurality of barcoded nucleic acid molecules formed by complementary base pairing of: (a) the capture sequence of the plurality of nucleic acid barcode molecules and (b) a capture handle sequence of an mRNA or DNA analyte comprising a nucleic acid sequence encoding at least a portion of the ABM. Some embodiments further include a sequencer or sequencing system for sequencing the first of the plurality of barcoded nucleic acid molecules.

Some of any of the embodiments of the first or second system further include an analysis engine.

Some of any of the embodiments of the first or second system further include a network.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual analyte carriers.

FIG. 2 shows an example of a microfluidic channel structure for the controlled partitioning of beads into discrete droplets.

FIG. 3 illustrates an example of a barcode carrying bead.

FIG. 4 illustrates another example of a barcode carrying bead.

FIG. 5 schematically illustrates an example microwell array.

FIG. 6 schematically illustrates an example workflow for processing nucleic acid molecules.

FIG. 7 schematically illustrates an example target reagent, having first and second target antigens conjugated, or coupled, to a core support (labeled “Strep”). In the schematic, the core is also shown as being coupled to a reporter oligonucleotide and a fluorophore. In some embodiments, the core may be coupled to the reporter oligonucleotide (but not the fluorescent molecule) or the fluorescent molecule (but not the reporter oligonucleotide), or both the reporter oligonucleotide and the fluorescent molecule. In some embodiments, the target reagent may include the core conjugated to the first and the second target antigen where both the core (conjugated to the first and second target antigen) and the reporter oligonucleotide are coupled to the fluorescent molecule.

FIG. 8 schematically illustrates an example target reagent, having first and second target antigens conjugated, or coupled, to a core support (labeled “Strep”), where the first and second target antigens are coupled to first and second reporter oligonucleotides, respectively. In the schematic, the core is also shown as being coupled to an, optional, fluorescent molecule.

FIG. 9 schematically illustrates an example target reagent, having first and second target antigens conjugated, or coupled, to a core support (labeled “Strep”), where the where the first and second target antigens are coupled to first and second fluorescent molecules.

FIG. 10 schematically illustrates combination of target reagents for use in the methods, or for inclusion in the compositions, kits, and systems herein. The combination includes: (i) a first target reagent having first and second target antigens conjugated, or coupled, to a core support (labeled “Strep”), where the first and second target antigens are coupled to first and second reporter oligonucleotides, respectively, and where the core support is coupled to an, optional, fluorescent molecule; (ii) a second target reagent including the first target antigen and a third reporter oligonucleotide where the first target antigen is coupled to a core support, and where the core is, optionally, coupled to a fluorescent molecule; and (iii) a third target reagent including the second target antigen and a fourth reporter oligonucleotide where the second target antigen is coupled to a core support, and where the core is, optionally, coupled to a fluorescent molecule

FIG. 11 schematically illustrates example labelling agents with nucleic acid molecules attached thereto.

FIG. 12A schematically shows an example of labelling agents. FIG. 12B schematically shows another example workflow for processing nucleic acid molecules. FIG. 12C schematically shows another example workflow for processing nucleic acid molecules.

FIG. 13 schematically shows another example of a barcode-carrying bead.

FIG. 14 shows an exemplary microfluidic channel structure for delivering barcode carrying beads to droplets.

FIG. 15 shows a computer system that is programmed or otherwise configured to implement methods provided herein.

### DETAILED DESCRIPTION OF THE DISCLOSURE

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

Headings, e.g., (a), (b), (i) etc., are presented merely for case of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a check swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition can be a physical container, compartment, or vessel, such as a droplet, a flowcell, a reaction chamber, a reaction compartment, a tube, a well, or a microwell. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### Overview

Provided herein are methods, compositions and systems for the characterization of ABMs, e.g., TCRs, BCR, Abs and antigen-binding fragments of Abs, produced by cells, e.g., T cells or B cells, using immune profiling technologies (e.g., single cell immune profiling). The ABMs identified and characterized by the methods may be multispecific, e.g, bispecific, in that they may have specificity for more than one target antigen. The identification of such multispecific ABMs by a highthroughput method offers the ability to more rapidly and reliably identify and/or select for improved therapeutics, e.g, improved antivirals or improved anti-cancer agents. For example, improved antiviral ABMs may be capable of binding to multiple infectious agents, e.g., viruses, thereby reducing production time and costs of antiviral ABMs. Improved anticancer ABMs may be identified that simultaneously bind a tumor and an immune effector cell to ensure killing of the tumor cell by the immune effector, e.g., cytotoxic T, cell, or that block multiple overlapping, or mutually exclusive, signaling pathways by binding molecules on a tumor cell's surface to suppress functions, e.g., growth and/or angiogenesis, that support tumor growth.

### Methods of the Disclosure

As described in more detail below, one aspect of the disclosure relates to new approaches and methods, compositions, kits, and systems for the characterization of antigen-binding molecules (ABMs), e.g., TCRs, BCR, Abs or antigen-binding fragments thereof. The methods, compositions and systems provided herein may characterize an ABM by identifying it as having a particular nucleic acid sequence(s) and/or as having particular amino acid sequence(s). The methods, compositions, kits, and systems provided herein may further, or alternatively, characterize an ABM as binding to and/or having affinity for one or more, e.g., first and second, target antigens.

In some embodiments, the ABM identified or characterized by the methods provided herein may be a BCR, an Ab, or an antigen-binding fragment thereof. The ABM identified or characterized by the methods herein may be an Ab having an Immunoglobulin (Ig) A (e.g., IgA1 or IgA2), IgD, IgE, IgG (e.g., IgG1, IgG2, IgG3 and IgG4) or IgM constant region. The ABM may be a fragment of the Ab that may be any naturally occurring, enzymatically obtainable, synthetic, or genetically engineered polypeptide or glycoprotein that specifically binds an antigen to form a complex. An ABM that is a fragment of an Ab may be one of: (i) Fab fragments; (ii) F(ab′)2 fragments; (iii) Fd fragments; (iv) Fv fragments; (v) single-chain Fv (scFv) molecules; (vi) sdAb fragments; and (vii) minimal recognition units consisting of the amino acid residues that mimic the hypervariable region of an antibody (e.g., an isolated complementarity determining region (CDR) such as a CDR3 peptide), or a constrained FWR3-CDR3-FWR4 peptide. Further, an antigen-binding fragment of an Ab may be an engineered molecule, such as a domain-specific antibody, single domain antibody, chimeric antibody, CDR-grafted antibody, diabody, triabody, tetrabody, minibody, nanobody (e.g., monovalent nanobodies, bivalent nanobodies, etc.), a small modular immunopharmaceutical (SMIP), or a shark immunoglobulin new antigen receptor (IgNAR) variable domain.

In other embodiments, the ABM identified or characterized in the methods provided herein may be a TCR. The TCR is a molecule found on the surface of T cells that is generally responsible for recognizing fragments of antigen as peptides bound to major histocompatibility complex (MHC) molecules. The TCR is generally a heterodimer of two chains, each of which is a member of the immunoglobulin superfamily, possessing an N-terminal variable (V) domain, and a C terminal constant domain. In humans, in 95% of T cells the TCR consists of an alpha (α) and beta (β) chain, whereas in 5% of T cells the TCR consists of gamma and delta (γ/δ) chains. This ratio can change during ontogeny and in diseased states as well as in different species. In certain instances, TCR may be a human TCR, or a mouse TCR. In certain instances, the TCR may be a sheep, cow, rabbit or chicken TCR. In some instances, the TCR may be a scFv-like soluble TCR.

In any of the embodiments herein, the ABM identified or characterized by the methods may be identified or characterized as being multispecific. A multispecific ABM may be an ABM having at least one antigen-binding site where the at least one antigen-binding site is specific for more than one antigen. A multispecific ABM may, alternatively, be an ABM having more than one antigen-binding site wherein a first antigen-binding site of the ABM binds a first antigen and at least a second binding site of the ABM that binds a different antigen. Multispecific ABMs may be in any format as described herein above and may further include, among others, bi-specific T-cell engager (BiTE), dual-affinity re-targeting protein (DART) and tandem diabody (TandAb) molecules. Other exemplary multispecific ABM formats are further described in Elshiaty, Mariam & Schindler, Hannah & Christopoulos, Petros. (2021). Principles and Current Clinical Landscape of Multispecific Antibodies against Cancer. International Journal of Molecular Sciences. 22. 5632. 10.3390/ijms22115632, which is hereby incorporated by reference.

The ABM, identified or characterized by the methods provided herein, may be so identified or characterized by its having bound to, or having binding affinity for, a first target reagent having first and second target antigens. The first and second target antigens of the first target reagent may be any antigens to which binding by an ABM is desirable. The first and/or second target antigen of the first target reagent may be an antigen or antigens associated with an infectious agent, such as a viral, bacterial, parasitic, protozoal or prion agent. A first and/or second target antigen associated with an infectious agent, wherein the infectious agent is a viral agent, may be an antigen or antigens of an influenza virus, a coronavirus, a retrovirus, a rhinovirus, or a sarcoma virus. A first and/or second target antigen of the first target reagent may be an antigen or antigens associated with a severe acute respiratory syndrome coronavirus 1 (SARS-COV-1), a SARS-COV-2, a Middle East respiratory syndrome coronavirus (MERS-COV)), human immunodeficiency virus (HIV), influenza, respiratory syncytial virus, or Ebola virus. Non-limiting examples of antigens of viral agents include corona virus spike(S) protein, influenza hemagglutinin protein, an HIV envelope protein or any other a viral glycoprotein. In other embodiments, the first and/or second target antigen of the first target reagent may be an antigen or antigens associated with a tumor or a cancer. Examples of antigens associated with a tumor or cancer include epidermal growth factor receptor (EGFR), CD38, platelet-derived growth factor receptor (PDGFR) alpha, insulin growth factor receptor (IGFR), CD20, CD19, CD47, ERBB2IP, TPS3. KRAS, MAGEAI, LC3A2, KIAA0368. CADPS2, CTSB or human epidermal growth factor receptor 2 (HER2). Other first and/or second target antigens associated with a tumor or cancer may be of one or more antigens that are checkpoint molecules. Checkpoint molecules include, but are not limited to, CD38, PD-1, CTLA-4, TIGIT, LAG-3, VISTA, TIM-3. Further, first and/or second target antigens of the first target reagent may be an antigen or antigens of one or more of: cytokines, GPCRs, cell-based co-stimulatory molecules, cell-based co-inhibitory molecules, ion channels, and growth factors. Further still, first and or second target antigens may be one or more antigens associated with a degenerative condition or disease (e.g., amyloid protein).

The first and/or second target antigen, in some embodiments, may be an antigen or antigens of any length, e.g., a length of at least about 20 amino acid residues, at least about 40 amino acid residues, at least about 60 amino acid residues, at least about 80 amino acid residues, at least about 100 amino acid residues, at least about 200 amino acid residues, at least about 300 amino acid residues, at least about 400 amino acid residues, at least about 500 amino acid residues, at least about 600 amino acid residues, at least about 700 amino acids, at least about 800 amino acid residues, at least about 900 amino acid residues, at least about 1000 amino acid residues, at least about 1100 amino acid residues, at least about 1200 amino acid residues, at least about 1300 amino acid residues, up to about 40 amino acid residues, up to about 60 amino acid residues, up to about 80 amino acid residues, up to about 100 amino acid residues, up to about 200 amino acid residues, up to about 300 amino acid residues, up to about 400 amino acid residues, up to about 500 amino acid residues, up to about 600 amino acid residues, up to about 700 amino acids, up to about 800 amino acid residues, up to about 900 amino acid residues, up to about 1000 amino acid residues, up to about 1100 amino acid residues, up to about 1200 amino acid residues, or up to about 1300 amino acid residues. The first and/or second target antigen may be a antigen or antigens that includes one domain, at least one domain, two domains, at least two domains, three domains, at least three domains, four domains, at least four domains, five domains, at least five domains, six domains, at least six domains, seven domains, at least seven domains, eight domains, at least eight domains, nine domains, at least nine domains, ten domains, at least ten domains, at least thirty domains, at least forty domains, at least fifty domains, at least sixty domains, at least seventy domains, at least eighty domains, at least ninety domains or at least one hundred domains. The first and/or second target antigen may be an antigen or antigens that includes at most two hundred domains, at most 175 domains, at most 150 domains, at most 125 domains, at most 100 domains, at most 75 domains, at most 50 domains, at most 25 domains, at most 20 domains, at most 15 domains, at most 10 domains, or at most 5 domains. The first and/or second target antigen may be a protein or peptide as expressed by a cell, e.g., full-length target antigen that may or may not include its leader sequence and may or may not have undergone a similar cell-processing step.

In some embodiments, the first and/or second target antigens of the first target reagent, to which binding by an ABM is desirable, may be or include first and second MHC molecules. In embodiments in which the first and/or second target antigen are first and/or second MHC molecules, the first and/or second MHC molecules may be complexed with first and second target antigenic peptides, respectively. The first and/or second target antigenic peptides may be peptides or peptide fragments of any antigen or antigens as described above, e.g., antigens described as suitable for inclusion in the first target reagent as the first and/or second target antigens. For example, the first and/or second target antigenic peptide that may be complexed with a first and/or second MHC molecule may be any peptide(s), or peptide fragment(s) of an antigen associated with one or more infectious agent, such as a viral, bacterial, parasitic, protozoal or prion agent. The first and second target antigenic peptides that may be complexed with the first and second MHC molecules may be peptides or peptide fragments of an antigen or antigens associated with a tumor or a cancer, e.g., growth factor receptor or transcription factor. Further, the first and/or second target antigenic peptides that may complexed with the first and second MHC molecules may be peptides or peptide fragments of any antigen or antigens associated with any degenerative condition or disease. Further yet, in some embodiments, the first, the second, or the first and the second MHC molecule may be complexed with a target lipid(s) or target small molecule(s).

In the methods of identifying or characterizing an ABM, (e.g., TCR, BCR, Ab or antigen-binding fragment thereof), as provided herein, a reaction mixture, or a portion thereof, may be partitioned into a plurality of partitions. The reaction mixture, or portion thereof, for partitioning in the methods may include a plurality of cells expressing ABMs and a plurality of target reagents. The plurality of cells expressing ABMs may be or include a plurality of B cells, a plurality of T cells, or a plurality of B and T cells. In instances in which the reaction mixture includes a plurality of B cells, a plurality of T cells, or a plurality of B cell and T cells, the plurality of cells may be have been enriched from a sample prior to inclusion in the reaction mixture for the partitioning. The enrichment for B cells, T cells or B and T cells for inclusion in the reaction mixture may be performed by any method, such as by labeling cells with a detectable moiety, e.g., fluorescent or magnetic marker (e.g., based on an expressed cell surface marker or another marker) and subjected to a FACS or MACS process to separate the B, T or T and T cells from other cells. In some embodiments, the expressed cell surface marker for enriching for B cells may be CD19. In other embodiments, the expressed cell surface marker for enriching for T cells may be CD3, CD4 and/or CD8.

The plurality of cells expressing ABMs for inclusion in the reaction mixture, whether or not enriched for B, T or B and T cells, may be from a sample of a subject, e.g., a vertebrate or a mammal such as a human or mouse (e.g., transgenic mouse). In some instances, the subject is a transgenic mouse having human HLA genes, human V(D)J genes or both. In instances in which the plurality of cells expressing ABMs is from a sample of the subject, the sample of the subject may have been obtained by biopsy, core biopsy, needle aspirate, or fine needle aspirate. The plurality of cells expressing ABMs for inclusion in the reaction mixture may be from a fluid sample of the subject, such as a blood sample. In instances in which the plurality of cells expressing ABMs for inclusion in the reaction mixture is from a sample of the subject, the sample may have been processed prior to its inclusion in the reaction mixture. The processing of the sample may include steps such as filtration, selective precipitation, purification, centrifugation, agitation, heating, and/or other processes. For example, a sample may be filtered to remove a contaminant or other materials. In some cases, cells and/or cellular constituents of a sample may be processed to separate and/or sort cells of different types, e.g., to separate B and/or T cells, as discussed herein (e.g., by FACS or MACS based on an expressed cell surface marker), from other cell types. A separation process may be a positive selection process, a negative selection process (e.g., removal of one or more cell types and retention of one or more other cell types of interest), and/or a depletion process (e.g., removal of a single cell type from a sample, such as removal of red blood cells from peripheral blood mononuclear cells). The subject, from whom the sample may have been obtained, may have been exposed to, expected to have been exposed, resistant to, suspected of being resistant to, or immunized against the first and/or second target antigen.

In instances in which the sample is obtained from a subject who has been immunized against the first and/or second target antigen, the subject may be a mouse, e.g., transgenic mouse having human HLA, human V(D)J or both human HLA and V(D)J genes. The subject, e.g., transgenic mouse, may have been immunized against the first and/or second target antigen via administration of the first and/or second target antigen, optionally followed by boosting with the first and/or second target antigen, e.g., same first and/or second target antigen as was initially administered. In an example schedule, a transgenic mouse may be immunized with the first and/or second target antigen on day 0, and administered booster immunizations on days 14, 28, 42 and 51.

The reaction mixture, or portion thereof, that may be partitioned into the plurality of partitions in the methods provided herein, may include a plurality of target reagents in addition to the plurality of, e.g., B and/or T, cells expressing ABMs.

The plurality of target reagents may include a first target reagent. The first target reagent may include a first target antigen and a second target antigen to which binding by an ABM, e.g., TCR, BCR, Ab or antigen-binding fragment of an Ab, may be desirable. The first target antigen and the second target antigen may be any of the target antigens described herein above. For example, in some embodiments, the first and or second target antigen may be one or more antigens associated with an infectious agent, such as a viral, bacterial, parasitic, protozoal or prion agent. The first and/or second target antigen may be an antigen or antigens associated with a tumor or a cancer, e.g., growth factor receptor or transcription factor. Further, the first and/or second target antigen may be an antigen or antigens associated with a degenerative condition or disease, or may be a cytokine or cytokines, or one or more of co-stimulatory molecule(s), co-inhibitory molecule(s), growth factor(s), or ion channel(s).

Moreover, in other embodiments, the first and/or second target antigens of the first target reagent may be may be or include first and second MHC molecules that may be complexed with first and second target antigenic peptides. In instances in which first and/or second target antigen of the first target reagent include an MHC molecule, e.g., first and/or second MHC molecule, the MHC molecule(s) may be or include an MHC class I or MHC class II molecule. In instances in which the first and/or second target antigens are, or include, an MHC class I molecule, the first and/or second target antigen may be, or include, a human MHC class I molecule, e.g., human leukocyte antigen (HLA)-A, HLA-B, HLA-C, HLA-E, HLA-F or HLA-G molecule. Examples of HLA-A molecules that may be, or may be included in, the first and/or second, target antigen of the first target reagent include HLA-A allele A*01:01, A*02:01, A*02:03, A*02:06, A*02:07, A*03:01, A*11:01, A*23:01, A*24:02, A*25:01, A*26:01, A*29:02, A*30:01, A*31:01, A*32:01, A*33:03, A*34:02, A*68:01, A*68:02, or A*74:01 molecules. Examples of HLA-B molecules that may be, or may be included in, the first and/or second target antigen of the first target reagent include HLA-B allele B*07:02, B*08:01, B*14:02, B*15:01, B*15:02, B*15:03, B*18:01, B*35:01, B*38:02, B*40:01, B*40:02, B*42:01, B*44:02, B*44:03, B*45:01, B*46:01, B*49:01, B*51:01, B*52:01, B*53:01, B*54:01, B*55:02, B*57:01 or B*58:01 molecules. Examples of HLA-C molecules that may be, or may be included in, the first and/or second target antigen of the first target reagent include HLA-C allele C*01:02, C*02:02, C*03:02, C*03:03, C*03:04, C*04:01, C*05:01, C*06:02, C*07:01, C*07:02, C*08:01, C*08:02, C*12:03, C*14:02, C*16:01, C*17:01 or C*18:01 molecules.

In other instances in which first and/or second target antigens of the first target reagent may be, or include, an MHC molecule, e.g., first and/or second MHC molecule, the MHC molecule(s) may be, or may include, one or more human MHC class II molecule, e.g., HLA-DP, HLA-DM, HLA-DOA, HLA-DOB, HLA-DQ or HLA-DR molecule. Examples of HLA-DR molecules that may be, or may be included in, the first and/or second target antigen of the first target reagent include HLA-DR allele DRB1*0101, DRB1*0301, DRB1*0401, DRB1*0701, DRB1*0801, DRB1*1101, DRB1*1301, DRB1*1501, DRB3*0101, DRB3*0202, DRB4*0101 or DRB5*0101 molecules. Examples of HLA-DP molecules that may be, or may be included in, the first and/or second target antigen of the first target reagent include HLA-DP allele DPA1*0103, DPA1*0202, DPAB1*0401 or DPAB1*0402 molecules. Examples of HLA-DQ molecules that may be, or may be included in, the first and/or second target antigen of the first target reagent include HLA-DQ allele DQA1*0101, DQB1*0301 or DQB1*0402 molecules.

In yet other instances in which first and/or second target antigens of the first target reagent may be, or include, an MHC molecule, e.g., first and/or second MHC molecule, the first and/or second MHC molecule may be, or include, an MHC class I-related protein (MR1) molecule or an antigen-presenting molecule CD1. Examples of human CD1 molecules include CD1a, CD1b, and CD1c molecules. The MR1, CD1, e.g., CD1a, CD1b and CD1c, molecules may be useful as MHC molecules in first and/or second target antigens of the first target reagent where the first and/or second MHC molecule is complexed with a lipid or small molecule.

In yet further instances in which first and/or second target antigens of the first target reagent include an MHC molecule, e.g., first and/or second MHC molecule, the MHC molecule(s) of the first and/or second target antigen may be, or include, a mouse MHC class I or MHC class II molecule. Examples of mouse MHC class I molecules include H-2K, H-2D, or H-2L molecules. Examples of mouse MHC class II molecules include a I-A or I-E molecules. Other mouse MHC molecules that may be, or may be included in, a first and/or second target antigen of the first target reagent include mouse MHC class 1b molecules, such as a Qa-2 or Qa-1 molecule.

As discussed earlier herein above, in embodiments in which the first and/or second target antigen include first and/or second MHC molecules, the first and/or second MHC molecules may be complexed with first and second target antigenic peptides. The first and second target antigenic peptides that may complexed with the first and second MHC molecules may be peptides or peptide fragments of any antigen or antigens to which binding by an ABM is desirable. For example, the first and/or second target antigenic peptide may be any peptide(s), or peptide fragment(s) of an antigen or antigens associated with one or more infectious agent, such as a viral, bacterial, parasitic, protozoal or prion agent. The first and second target antigenic peptides that may be complexed with the first and second MHC molecules may be peptides or peptide fragments of any antigen or antigens associated with a tumor or a cancer, e.g., growth factor receptor or transcription factor. Further, the first and/or second target antigenic peptides that may complexed with the first and second MHC molecule may be peptides or peptide fragments of any antigen or antigens associated with any degenerative condition or disease. Further yet, in some embodiments, the first, the second, or the first and the second MHC molecule may be complexed with a target lipid(s) or target small molecule(s).

A target antigenic peptide, e.g., first and/or second target antigenic peptide, that may be complexed with a first and/or second MHC molecule, may be of any suitable length, e.g., of a length selected for optimal binding to a particular MHC molecule's, e.g., specific allele's, peptide binding groove. The target antigenic peptide, e.g., first and/or second target antigenic peptide, that may be complexed with a first and/or second MHC molecule may be at least 5, 6, 7, 8, 9, 10, 11, 12, 13, 14, 15, 16, 17, 18, 19, 20, 21, 22, 23, 24, 25, 26, 27, 28, 29, or 30 amino acids in length. The target antigenic peptide that may be complexed with the first and/or second MHC molecule may be at most about 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 amino acids in length. The target antigenic peptide that may be complexed with a first and/or second MHC molecule may be between about 5 and about 35, between about 6 and about 34, between about 7 and about 33, between about 8 and about 32, between about 9 and about 31, between about 10 and about 30, between about 11 and about 29, between about 12 about and 28, between about 13 and 27, between about 14 and 26, between about 15 and 25, between about 16 and 24, between about 17 and 23, or between about 18 and 22 amino acids in length.

A peptide bound to an MHC class I molecule may be between about 6 to 12 amino acids in length, e.g., between about 7 to 11 amino acids in length, or between about 8 to 10 amino acids in length. A target antigenic and/or control peptide bound to an MHC class II molecule may be between about 5 to 35 amino acids in length, between about 10 to 30 amino acids in length, between about 15 to 25 amino acids in length, or between about 13 and 25 amino acids in length.

The target antigenic peptide that may be complexed with a first/and or second MHC molecule may be a peptide having a sequence selected/derived from a protein or peptide by any, e.g., computational prediction, method. A computational prediction method for selection of the antigenic target peptide from the sequence of the protein or peptide, may be one based on an artificial learning system that uses, e.g., motif-based methods, machine learning methods, semi-supervised machine learning methods, or combinations thereof. A motif-based method for target antigenic peptide selection may be one based on a position weight matrix to model a gapless multiple sequence alignment of peptides. A Machine learning method may be one based on artificial neural networks. Examples of neural networks that may be used to select a target antigenic peptide from a target antigen include Pepdist, MHCflurry, NetMHC, NetMHCpan, NetMHCpan4.0, MixMHCpred 2.0.1, NetMHCcons 1.1, NetMHCII, NetMHCIIpan and PUFFIN.

The first target reagent of the plurality of target reagents, including the first and the second target antigens, may further include a core support. The first and the second target antigen of the first target reagent may be coupled to the core support. In some instances, the first and second target antigen may be biotinylated. In such instances, the core support may be streptavidin, avidin, deglycosylated avidin, traptavidin, tamavidin, xenavidin, or bradavidin.

The first target reagent of the plurality of target reagents, including the first and the second target antigen, may further, or alternatively, be coupled to one or more additional molecules. The target reagent of the plurality of target reagents may be coupled to a first a reporter oligonucleotide or to the first reporter oligonucleotide and an additional reporter oligonucleotide. The target reagent may be coupled to a first detectable label, e.g., first fluorescent molecule, or to the first and an additional detectable label, e.g., first and additional fluorescent molecule. The target reagent may be coupled to a first reporter oligonucleotide and a first detectable label, e.g, fluorescent molecule. The target reagent may be coupled to a first reporter oligonucleotide, an additional reporter oligonucleotide and a first detectable label, e.g, fluorescent molecule. The target reagent may be coupled to a first reporter oligonucleotide, an additional reporter oligonucleotide, a first detectable label, e.g., fluorescent molecule, and an additional detectable label, e.g., fluorescent molecule.

The first target reagent, in some embodiments, may include the first target antigen, the second target antigen, and the first reporter oligonucleotide. In such embodiments, the first reporter oligonucleotide may be coupled to the first or the second target antigen. In other such embodiments, the first target reagent may include (i) the first and the second target antigen coupled to the core support and (ii) the first reporter oligonucleotide coupled to any of the (a) first target reagent, (b) second target reagent or (c) core support. The first reporter oligonucleotide, in such embodiments, may include a first reporter barcode sequence. The first reporter barcode sequence may identify the first target reagent. The first reporter oligonucleotide may further include a capture handle sequence and may include one or more further functional, e.g, UMI or primer binding, sequences.

The first target reagent, in some embodiments, may include the first target antigen, the second target antigen, the first reporter oligonucleotide and an additional reporter oligonucleotide. In such embodiments, the first target reagent may include (i) the first target antigen coupled to the first reporter oligonucleotide and (ii) the second target antigen coupled to the additional reporter oligonucleotide. In other such embodiments, the first target reagent may include the first target antigen coupled to the first reporter oligonucleotide, the second target antigen coupled to the additional reporter oligonucleotide, and both the first target antigen (coupled to the first reporter oligonucleotide) and the second target antigen (coupled to the additional reporter oligonucleotide) coupled to the core support. The first reporter oligonucleotide, in such embodiments, may include a first reporter barcode sequence. The first reporter barcode sequence may identify the first target antigen. The additional reporter oligonucleotide, in such embodiments, may further include an additional reporter barcode sequence. The additional reporter barcode sequence may identify the second target antigen. The first and the additional reporter oligonucleotide may further include capture handle sequences, e.g., same or different capture handle sequences. The first and the additional reporter oligonucleotides may futher include one or more further functional, e.g, UMI or primer binding, sequences.

The first target reagent, in yet other embodiments, may include the first target antigen, the second target antigen and a first fluorescent molecule. In such embodiments, the first fluorescent molecule may be coupled to the first target antigen or the second target antigen. In other such embodiments, the first target reagent may include (i) the first and the second target antigen coupled to a core support and (ii) the first fluorescent molecule coupled to any of the (a) first target antigen, (b) second target antigen or (c) core support.

The first target reagent, in yet other embodiments, may include the first target antigen, the second target antigen, the first fluorescent molecule and an additional fluorescent molecule. In such embodiments, the first fluorescent molecule may be coupled to the first target antigen and the additional fluorescent molecule may be coupled to the second target antigen. In other such embodiments, the first target reagent may include (i) the first target antigen coupled to the first fluorescent molecule, (ii) the second target antigen coupled to the additional fluorescent molecule, and (iii) both the first target antigen (coupled to the first fluorescent molecule) and the second target antigen (coupled to the additional fluorescent molecule) coupled to the core support.

In yet other embodiments, the first target reagent may include the first target antigen, the second target antigen, the first reporter oligonucleotide and the first fluorescent molecule. In such embodiments, the first reporter oligonucleotide may be coupled to the first or second target antigen and the first fluorescent molecule may be coupled to the first or second target antigen. In other such embodiments, the target reagent may include the first target antigen and the second target antigen coupled to the core support, where the first reporter oligonucleotide and/or the first fluorescent molecule are coupled to any of the first target antigen, the second target antigen, or (separately) the first and the second target antigen. In yet other such embodiments, the first target reagent may include, coupled to the core support, the: (i) first target antigen, (ii) second target antigen, (iii) first reporter oligonucleotide and (iv) first fluorescent molecule. In a further such embodiment, the first target reagent may include, coupled to the fluorescent molecule, the: (i) the core support (coupled to the first and second target antigens) and (iii) the first reporter oligonucleotide. The first reporter oligonucleotide, in such embodiments, may include a first reporter barcode sequence. The first reporter barcode sequence may identify the first target reagent. The first reporter oligonucleotide may further include a capture handle sequence and may include one or more further functional, e.g, UMI or primer binding, sequences.

The first target reagent, in yet a further embodiment, may include the first target antigen, the second target antigen, the first reporter oligonucleotide, the additional reporter oligonucleotide and the first fluorescent molecule. In such embodiments, the first target reagent may include (i) the first reporter oligonucleotide coupled to the first target antigen, (ii) the additional reporter oligonucleotide coupled to the second target antigen and (iii) the first fluorescent molecule coupled to the first target antigen (coupled to the first reporter oligonucleotide) or second target antigen (coupled to the additional reporter oligonucleotide). In other such embodiments, the first target reagent may include (i) the first target antigen coupled to the first reporter oligonucleotide, (ii) the second target antigen coupled to the additional reporter oligonucleotide, (iii) the first target antigen (coupled to the first reporter oligonucleotide) and the second target antigen (coupled to the additional reporter oligonucleotide) coupled to the core support and (iv) the first fluorescent molecule coupled to the core support, the first target antigen (coupled to the first reporter oligonucleotide) or the second target antigen (coupled to the additional reporter oligonucleotide). The first reporter oligonucleotide, in such embodiments, may include a first reporter barcode sequence. The first reporter barcode sequence may identify the first target antigen. The additional reporter oligonucleotide, in such embodiments, may further include an additional reporter barcode sequence. The additional reporter barcode sequence may identify the second target antigen. The first and the additional reporter oligonucleotide may further include capture handle sequences, e.g., same or different capture sequences. The first and the additional reporter oligonucleotides may further include one or more further functional, e.g, UMI or primer binding, sequences.

The first target reagent, in a yet further embodiment, may include the first target antigen, the second target antigen, the first reporter oligonucleotide, the first fluorescent molecule, and the additional fluorescent molecule. In such embodiments, the first target reagent may include (i) the first target antigen coupled to the first fluorescent molecule, (ii) the second target antigen coupled to the additional fluorescent molecule, and (iii) the first reporter oligonucleotide coupled to either first target antigen (coupled to the first fluorescent molecule) or the second target antigen (coupled to the additional fluorescent molecule). In other such embodiments, the first target reagent may include (i) the first target antigen coupled to the first fluorescent molecule, (ii) the second target antigen coupled to the additional fluorescent molecule, (iii) the first target antigen (coupled to the first fluorescent molecule) and the second target antigen (coupled to the additional fluorescent molecule) coupled to the core support and (iv) the first reporter oligonucleotide coupled to the core support, the first target antigen (coupled to the first fluorescent molecule) or the second target antigen (coupled to the additional fluorescent molecule). The first reporter oligonucleotide, in such embodiments, may include a first reporter barcode sequence. The first reporter barcode sequence may identify the first target reagent. The first reporter oligonucleotide may further include a capture handle sequence and may include one or more further functional, e.g, UMI or primer binding, sequences.

The first target reagent, in a still further embodiment, may include the first target antigen, the second target antigen, the first reporter oligonucleotide, the additional reporter oligonucleotide, the first fluorescent molecule, and the additional fluorescent molecule. In such embodiments, the first target reagent may include (i) the first target antigen coupled to the first reporter oligonucleotide and the first fluorescent molecule, and (ii) the second target antigen coupled to the additional oligonucleotide and the additional fluorescent molecule. In other such embodiments, the first target reagent may include, coupled to the core support: (i) the first target antigen coupled to the first reporter oligonucleotide and the first fluorescent molecule; and (ii) the second target antigen coupled to the additional reporter oligonucleotide and the additional fluorescent molecule. The first reporter oligonucleotide, in such embodiments, may include a first reporter barcode sequence. The first reporter barcode sequence may identify the first target antigen. The additional reporter oligonucleotide, in such embodiments, may further include an additional reporter barcode sequence. The additional reporter barcode sequence may identify the second target antigen. The first and the additional reporter oligonucleotide may further include capture handle sequences, e.g., same or different capture sequences. The first and the additional reporter oligonucleotides may further include one or more further functional, e.g, UMI or primer binding, sequences.

In embodiments in which the first target reagent of the plurality of target reagents includes a fluorescent molecule, e.g., include a first and/or additional fluorescent molecule, the first and/or additional fluorescent molecule may be any of the following: Alexa Fluor 350, DyLight 405, Alexa Fluor 405, Pacific Blue, Alexa Fluor 405, Alexa Fluor 430, Alexa Fluor 488, fluorescein isothiocyanate (FITC), Alexa Fluor 532, Alexa Fluor 546, DyLight 550, phycocrythrin (PE), allophycocyanin (APC), Alexa Fluor 555, Alexa Fluor 561, Alexa Fluor 568, Alexa Fluor 594, Alexa Fluor 610, Alexa Fluor 633, Alexa Fluor 635, Alexa Fluor 647, DyLight 650, peridinin chlorophyll protein (PerCP), Alexa Fluor 660, Alexa Fluor 680, Alexa Fluor 700, Alexa Fluor 750, Alexa Fluor 790, Coumarin, borondipyrromethene (BODIPY), Pacific Green, Oregon Green, cyanine (Cy) 3, Cy5, Pacific Orange, PE-Cy7, PerCP-Cy5.5, Tetramethylrhodaminie (TRITC), Texas Red, StarBright Violet 440, StarBright Violet 515, StarBright 610, StarBright Violet 670 or StarBright Blue 700. The first and the additional fluorescent molecule in any of the embodiments herein may have different detectable signals.

In instances in which the first target reagent of the plurality of target reagents includes a fluorescent molecule, e.g., first and/or additional fluorescent molecule, cells of the plurality of cells that may be sorted, or selected, for having bound the first target reagent based on their comprising the fluorescent, e.g., first and/or additional fluorescent, molecule. For example, cells of the plurality of cells may be selected or sorted for having bound to the first target reagent based on their comprising the first fluorescent molecule in instances in which the first target reagent included the first target antigen, the second target antigen and: (i) the first fluorescent molecule; (ii) the first fluorescent molecule and the additional fluorescent molecule; (iii) the first reporter oligonucleotide and the first fluorescent molecule; (iv) the first reporter oligonucleotide, the additional reporter oligonucleotide and the first fluorescent molecule; (v) the first reporter oligonucleotide, the first fluorescent molecule, and the additional fluorescent molecule; or (vi) the first reporter oligonucleotide, the additional reporter oligonucleotide, the first fluorescent molecule, and the additional fluorescent molecule. For other example, cells of the plurality of cells may be selected or sorted for having bound to the first target reagent based on their comprising the additional fluorescent molecule in instances in which the first target reagent included the first target antigen, the second target antigen and: (i) the first fluorescent molecule and an additional fluorescent molecule, (ii) the first reporter oligonucleotide, the first fluorescent molecule, and the additional fluorescent molecule or (iii) the first reporter oligonucleotide, the additional reporter oligonucleotide, the first fluorescent molecule, and the additional fluorescent molecule. In yet another example, cells of the plurality of cells may be selected or sorted for having bound to the first target reagent based on their comprising the first and the additional fluorescent molecule in instances in which the first target reagent included the first target antigen, the second target antigen and: (i) the first fluorescent molecule and an additional fluorescent molecule, (ii) the first reporter oligonucleotide, the first fluorescent molecule, and the additional fluorescent molecule or (iii) the first reporter oligonucleotide, the additional reporter oligonucleotide, the first fluorescent molecule, and the additional fluorescent molecule.

Further, and in yet other instances, cells may be sorted for having bound the first target reagent and for having bound second and/or third target reagents that may be included in the plurality of target reagents. In these yet other such instances, the second target reagent may include the first target antigen and a second fluorescent molecule and the third target reagent may include the second target antigen and a third fluorescent molecule. In these yet other such instances, the cells may be sorted or selected for comprising the first and/or further fluorescent molecule as described in the immediately preceding paragraph and may further be sorted or selected for additionally comprising the second and/or third fluorescent molecule. A sorted cell comprising the first and/or second fluorescent molecule, and further comprising the second and third fluorescent molecule may be characterized as being multispecific for the first and the second target antigen.

The reaction mixture, or portion thereof, that may be partitioned into the plurality of partitions in the methods provided herein, may include, in addition to the plurality of target reagent and plurality of cells expressing ABMs, a plurality of additional labelling agents. In some embodiments, the additional labeling agents are configured to bind or otherwise couple to one or more cell-surface features of a cell, e.g., cell expressing an AMB. In some embodiments, such additional labeling agents can be used to characterize cells and/or cell features. In some embodiments, one or more of the additional labelling agents comprise a detectable label, e.g., a detectable label described herein. In some embodiments, one or more of the additional labelling agents comprise a reporter oligonucleotide. In some embodiments, reporter oligonucleotides of the one or more additional labeling agents have different primer sequences, e.g., different sequencing primer sequences than reporter oligonuncleotides coupled to the target reagents. In some embodiments, the cells expressing ABMs are contacted with the target reagents, then with the additional labelling agents.

The partitioning of the reaction mixture, or portion thereof, may provide a partition that includes: (i) a cell, e.g., B or T cell, expressing an ABM, that had bound the first target reagent and (ii) the first target reagent. The partition provided by the partitioning may include the cell, e.g., B or T cell, expressing the ABM, where the cell is bound to the first target reagent. In some embodiments, the cell in the provided partition may be a B cell and the B cell in the provided partition may be bound to the first target reagent, e.g., by a BCR expressed by the B cell being bound to the first target reagent. In other embodiments, in which the cell in the provided partition is a B cell, the B cell in the provided partition may be bound to the first target reagent by tethering of a secreted Ab expressed by the B cell to the B cell, e.g., via a capture agent such as a polypeptide (e.g., Ab) or aptamer that binds/couples to both the cell and its secreted Ab, where the secreted Ab is bound to the first reagent. In yet other embodiments, the cell in the provided partition may be a T cell and the T cell in the provided partition may be bound to the first target reagent, e.g., by a TCR expressed by the T cell being bound to the first target reagent. While the partitioning may provide a partition including a cell expressing an ABM where the cell is bound to the first reagent, the cell expressing the ABM need not be, or remain bound, to the first reagent in the provided partition. For example, the cell expressing the ABM may have bound to the first target reagent in the reaction mixture, and, upon partitioning in the provided partition, became no longer bound to first target reagent.

The partition provided at the step of partitioning, including the (i) cell expressing the ABM, e.g., B or T cell, that had bound the first target reagent and (ii) the first target reagent, may further include (iii) a plurality of nucleic acid barcode molecules. The plurality of nucleic acid barcode molecules may have a partition-specific barcode sequence. The plurality of nucleic acid barcode molecules may further have a capture sequence. In some embodiments, the capture sequence of a first of the plurality of nucleic acid barcode molecules, is configured to couple to an mRNA or DNA analyte of the cell expressing the ABM, e.g., B or T cell, in the provided partition. In such embodiments, the capture sequence of the first nucleic acid barcode molecule includes a polyT sequence, a random sequence, or a sequence complementary to a gene-specific sequence, e.g., sequence of an immunoglobulin variable or constant region, or B cell receptor variable or constant region or T cell receptor variable or constant region. Alternatively, the capture sequence of the first nucleic acid barcode molecule may be configured to couple to non-templated nucleotides appended to a cDNA reverse transcribed, by a reverse transcriptase having terminal transferase activity, from an mRNA analyte of the cell expressing the ABM, e.g. B or T cell, in the provided partition. In embodiments in which the capture sequence of the first nucleic acid barcode molecule is configured to couple to non-templated nucleotides appended to a cDNA reversed transcribed from the mRNA analyte, the mRNA analyte may be reversed transcribed to the cDNA using a polyT primer, a primer of random sequence or a primer complementary to a gene-specific sequence as discussed above. During reverse transcription, the reverse transcriptase, via its terminal transferase activity, may append one or more non-templated nucleotides, e.g., cytosines, to the cDNA. In instances in which the non-templated nucleotides appended to the cDNA are cytosines, the capture sequence of the nucleic acid barcode molecule may include one or more guanines. The first of the plurality of nucleic acid barcode molecules, in addition to the partition-specific barcode sequence and capture sequence, may further include one or more functional sequences, such as a unique molecule identifier (UMI), a template switch sequence, a sequencer attachment sequence, a sequencing primer sequence, an amplification primer sequence, or the complements thereof.

Barcoded nucleic acid molecules may be generated in the methods provided herein. The barcoded nucleic acid molecules may be generated in the provided partition, e.g., provided partition including the cell expressing the ABM, the first target reagent and the plurality of nucleic acid barcode molecules. The barcoded nucleic acid molecules may, alternatively, be generated in bulk. The barcoded nucleic acid polynucleotides may be generated in bulk by: (i) coupling of the capture sequence(s) of nucleic acid barcode molecule(s) to sequence(s) of mRNA, cDNA or DNA analytes of the cells expressing ABMs in their provided partitions and (ii) pooling, from a plurality of partitions, the nucleic acid barcode molecules coupled to the mRNA, cDNA, DNA analytes of the cells expressing ABMs.

The generated barcoded nucleic acid molecule may include a first barcoded nucleic acid molecule. The first barcoded nucleic acid molecule may include a nucleic acid sequence encoding at least a portion of the ABM expressed by the cell or reverse complement thereof and the partition-specific barcode sequence or reverse complement thereof. This generated first barcoded nucleic acid molecule may identify the ABM, e.g., by sequence, expressed by the cell that had been in the provided partition. The identification of the ABM from the first barcoded nucleic acid polynucleotide may be performed by a step of sequencing the generated first barcoded nucleic acid polynucleotide.

In embodiments in which the ABM is a BCR or an Ab or an antigen-binding fragment of an Ab, the nucleic acid sequence may encode at least a portion of a variable heavy (VH) or variable light (VL) chain of the BCR, Ab or antigen-binding fragment of the Ab. The sequencing of the first barcoded nucleic acid polynucleotide may determine the sequence encoding the at least a portion of the variable heavy (VH) or variable light (VL) chain of the BCR, Ab or antigen-binding fragment of the Ab. For example, it may determine sequence encoding a VH complementarity determining region (CDR)1, VH CDR2, VH CDR3, or VL CDR1, VL CDR2 or VL CDR3 of the BCR, Ab or antigen-binding fragment of the Ab. For other example, it may determine a sequence encoding a VH framework (FW) 1, FW2, FW3, FW4 or VL FW1, VL FW2, VL FW3 or VL FW4 of the BCR, Ab or antigen-binding fragment of the Ab. From the determined sequence encoding at least a portion of the BCR, Ab or antigen-binding fragment of the Ab, at least a portion of the amino acid sequence encoded by the determined nucleic acid sequence may be determined.

In embodiments in which the ABM is a TCR, the nucleic acid sequence may encode at least a portion of a TCR alpha chain, a TCR beta chain, a TCR delta chain or a TCR gamma chain. In such embodiments, the determined nucleic acid sequence may determine a sequence encoding any, or a portion, of a TCR alpha chain variable region, TCR beta chain variable region, TCR delta chain variable region or TCR gamma chain variable region of the TCR. It may determine a sequence encoding one or more CDRs (e.g., CDR1, CDR2, and CDR3), or hypervariable regions, in the variable regions of the TCR. From the determined sequence encoding at least a portion of the TCR, at least a portion of the amino acid sequence encoded by the determined nucleic acid sequence may be determined.

In certain embodiments of the methods herein, a further barcoded nucleic acid molecule encoding at least a second portion of the ABM expressed by the cell, may also be generated. The further barcoded nucleic acid molecule may include a nucleic acid sequence encoding at least a second portion of the ABM expressed by the cell or reverse complement thereof and the partition-specific barcode sequence or reverse complement thereof.

In instances in which the ABM is a BCR or an Ab or an antigen-binding fragment of an Ab, and in which the first and the further barcoded nucleic acid molecules are generated, the first barcoded nucleic acid molecule may include a sequence encoding at least a portion of a VH chain, e.g., VH CDR1, VH CDR2, VH CDR3, VH FW1, VH FW2, VH FW3 and/or VH FW4, and the further barcoded nucleic acid molecule may include a sequence encoding at least a portion of a VL chain, e.g., VL CDR1, VL CDR2, VL CDR3, VL FW1, VL FW2, VL FW3 and/or VL FW4, of the ABM expressed by the B cell that had been in the provided partition. In other instances, the first barcoded nucleic acid molecule may include a sequence encoding at least a portion of a VL chain, e.g., VL CDR1, VL CDR2, VL CDR3, VL FW1, VL FW2, VL FW3 and/or VL FW4, and the further barcoded nucleic acid polynucleotide may include a sequence encoding at least a portion of a VH chain, e.g., VH CDR1, VH CDR2, VH CDR3, VH FW1, VH FW2, VH FW3 and/or VH FW4 of the ABM expressed by a B cell that had been in the provided partition. In yet other examples, both the first and the further barcoded nucleic acid molecules may include a sequence encoding at least a portion of a VH chain, e.g., VH CDR1, VH CDR2 or VH CDR3, VH FW1, VH FW2, VH FW3 and/or VH FW4, or both the first and the additional barcoded nucleic acid molecules may include a sequence encoding at least a portion of a VL chain, e.g., VL CDR1, VL CDR2 or VL CDR3, VL FW1, VL FW2, VL FW3 and/or VL FW4, of the ABM expressed by a B cell that had been in the provided partition.

In instances in which the ABM is a TCR, and in which the first and the further barcoded nucleic acid molecules are generated, the first barcoded nucleic acid molecule may include a sequence encoding at least a portion of TCR alpha chain and the further barcoded nucleic acid molecule may include a sequence encoding at least a portion of a TCR beta chain of a TCR expressed by the T cell that had been in the provided partition. In such instances, the first barcoded nucleic acid molecule may include a sequence encoding at least a portion of TCR alpha chain variable region (e.g, CDR1, CDR2 and/or CDR3) and the further barcoded nucleic acid molecule may include a sequence encoding at least a portion of a TCR beta chain variable region (e.g., CDR1, CDR2 and/or CDR3) of the TCR expressed by the T cell that had been in the provided partition. In other instances, the first barcoded nucleic acid molecule may include a sequence encoding at least a portion of TCR gamma chain and the further barcoded nucleic acid molecule may include a sequence encoding at least a portion of a TCR delta chain of the TCR expressed by the T cell that had been in the provided partition. In such instances, the the first barcoded nucleic acid molecule may include a sequence encoding at least a portion of TCR gamma chain variable region (e.g, CDR1, CDR2 and/or CDR3) and the further barcoded nucleic acid molecule may include a sequence encoding at least a portion of a TCR delta chain variable region (e.g., CDR1, CDR2 and/or CDR3) of the TCR expressed by the T cell that had been in the provided partition. In some other instances, both the first and the further barcoded nucleic acid molecule may encode at least a portion of a TCR alpha chain (e.g., TCR alpha chain variable region, e.g., TCR alpha chain variable region CDR1, CDR2 and/or CDR3), or both the first and the further barcoded nucleic acid molecule may encode at least a portion of a TCR beta chain (e.g., TCR beta chain variable region, e.g., TCR beta chain variable region CDR1, CDR2 and/or CDR3), or both the first and the further barcoded nucleic acid molecule may encode at least a portion of a TCR gamama chain (e.g., TCR gamma chain variable region, (e.g., TCR gamma chain variable region CDR1, CDR2 and/or CDR3), or both the first and the further barcoded nucleic acid molecule may encode at least a portion of a TCR delta chain (e.g., TCR delta chain variable region, e.g., TCR delta chain variable region CDR1, CDR2 and/or CDR3) of the TCR expressed by the T cell that had been in the provided partition.

In some embodiments of the methods of the methods provided herein, a second barcoded nucleic acid molecule, e.g., having sequences other than those encoding at least a portion of an ABM, may be generated. In such embodiments, the first target reagent of the plurality of target reagents may have included the first reporter oligonucleotide. For example, in such embodiments, the first target reagent may have included the first target antigen, the second target antigen, and: (i) the first reporter oligonucleotide; (ii) the first reporter oligonucleotide and the additional reporter oligonucleotide; (iii) the first reporter oligonucleotide and the first fluorescent molecule; (iv) the first reporter oligonucleotide, the additional reporter oligonucleotide and the first fluorescent molecule; (v) the first reporter oligonucleotide, the first fluorescent molecule, and the additional fluorescent molecule; or (vi) the first reporter oligonucleotide, the additional reporter oligonucleotide, the first fluorescent molecule, and the additional fluorescent molecule. In these embodiments, the plurality nucleic acid molecules may include a second nucleic acid barcode molecule, e.g., having a capture sequence capable of coupling to the capture handle sequence of the first reporter oligonucleotide and a partition-specific barcode sequence. The second barcoded nucleic acid molecule generated in these embodiments may include a sequence of the first reporter oligonucleotide, e.g., first reporter barcode sequence, or a reverse complement thereof, and the partition-specific barcode sequence or a reverse complement thereof. In these embodiments, the second generated barcoded nucleic acid molecule may characterize the ABM, e.g., TCR, BCR, Ab or antigen-binding fragment of the Ab, expressed by the cell, e.g., B or T cell, as a candidate ABM having specificity for both the first and the second target antigen of the first target reagent.

In some other embodiments of the methods provided herein, an additional barcoded nucleic acid molecule may be generated. In such embodiments, the first target reagent of the plurality of target reagents may have included the first reporter oligonucleotide and an additional reporter oligonucleotide. In these embodiments, the first target reagent may have included the first target antigen, the second target antigen and: (i) the first reporter oligonucleotide and the additional reporter oligonucleotide; (ii) the first reporter oligonucleotide, the additional reporter oligonucleotide and the first fluorescent molecule; or (iii) the first reporter oligonucleotide, the additional reporter oligonucleotide, the first fluorescent molecule, and the additional fluorescent molecule. In these embodiments, embodiments in which the first target antigen includes the additional reporter oligonucleotide and in which an additional barcoded nucleic acid molecule may be generated, the plurality nucleic acid molecules may include an additional nucleic acid barcode molecule. The additional nucleic acid molecule of the plurality of nucleic acid molecules may include a capture sequence, configured to couple to the capture handle sequence of the additional reporter oligonucleotide, and a partition-specific barcode sequence. The generated additional barcoded nucleic acid molecule may include a sequence of the additional reporter oligonucleotide, e.g., additional reporter barcode sequence, or a reverse complement thereof, and the partition-specific barcode sequence or a reverse complement thereof. In these embodiments, the additional generated barcoded nucleic acid molecule may characterize the ABM, e.g., TCR, Ab or antigen-binding fragment of the Ab, expressed by the cell, e.g., B or T cell, as a candidate ABM having specificity for both the first and the second target antigen of the first target reagent.

The characterization of the ABM expressed by the cell as a candidate ABM having specificity for both the first and the second target antigen of the first target reagent, may refer to a characterization of the ABM as having specificity for the first target antigen, the second target antigen, or both the first and the second target antigen of the first target reagent. In instances in which a B cell was in the provided partition, the characterization of the ABM as a candidate ABM may refer to the characterization of a BCR, an Ab or antigen-binding fragment of an Ab expressed by the B cell as being specific for the first target antigen, the second target antigen, or both the first and the second target antigen of the first target reagent. In instances in which a T cell that was in the provided partition, the characterization of the ABM as a candidate ABM may refer to the characterization of a TCR expressed by the T cell as being specific for the first target antigen, the second target antigen, or both the first and the second target antigen of the first target reagent.

To further characterize the candidate ABM as having specificity for the first target antigen, the second target antigen, or both the first and the second target antigen, the plurality of target reagents may further include in certain embodiments second and third target reagents. In instances in which the plurality of target reagents further include this certain embodiment of the second and the third target reagents, the second target reagent may include the first target antigen and a second reporter oligonucleotide. The second reporter oligonucleotide of the second target reagent may include a second reporter barcode sequence and may further include a capture handle sequence. The second reporter oligonucleotide's reporter barcode sequence may identify the second target reagent (and the first target antigen included therein). The second reporter oligonucleotide's capture handle sequence may be configured to couple to a capture sequence of a nucleic acid molecule of a plurality of nucleic acid molecules of a provided partition. The second reporter oligonucleotide may further include functional sequences as discussed herein. The first target antigen of the second target reagent may be coupled to a core support. In instances in which the first target antigen of the second target reagent is coupled to a core support, the first target antigen may be biotinyated and the core support may include any of streptavidin, avidin, deglycosylated avidin, traptavidin, tamavidin, xenavidin, or bradavidin.

In instances in which the plurality of target reagent include this certain embodiment of the second and third target reagents, the third target reagent may include the second target antigen and a third reporter oligoucleotide. The third reporter oligonucleotide of the third target reagent may include a third reporter barcode sequence and may further include a capture handle sequence. The third reporter oligonucleotide's reporter barcode sequence may identify the third target reagent (and the second target antigen included therein). The third reporter oligonucleotide's capture handle sequence may be configured to couple to a capture sequence of a nucleic acid molecule of a plurality of nucleic acid molecules of a provided partition. The third reporter oligonucleotide may further include functional sequences as discussed herein. The second target antigen of the third target reagent may be coupled to a core support. In instances in which the second target antigen of the third target reagent is coupled to a core support, the second target antigen may be biotinyated and the core support may include any of streptavidin, avidin, deglycosylated avidin, traptavidin, tamavidin, xenavidin, or bradavidin.

In instances in which the plurality of target reagents further include this certain embodiment of the second and the third target reagents, third and fourth barcoded nucleic acid molecules may generated, e.g., generated in addition to the first barcoded nucleic acid molecule (having a nucleic acid sequence encoding at least a portion of the ABM expressed by the cell or reverse complement thereof and the partition-specific barcode sequence or reverse complement thereof), second barcoded nucleic acid molecule (having a sequence of the first reporter oligonucleotide or a reverse complement thereof and the partition-specific barcode sequence or reverse complement thereof) and/or additional barcoded nucleic acid molecule (having a nucleic acid sequence the additional reporter oligonucleotide or reverse complement there and the partition-specific barcode sequence or reverse complement thereof). The third generated barcoded nucleic acid molecule may include the sequence of the second reporter oligonucleotide (e.g., that identifies the second target reagent, which includes the first target antigen) or a reverse complement thereof and the partition-specific barcode sequence or reverse complement thereof. The fourth generated barcoded nucleic acid molecule may include the sequence of the third reporter oligonucleotide (e.g,. that identifies the third target reagent, which includes the second target antigen) or a reverse complement thereof and the partition-specific barcode sequence or reverse complement thereof.

In instances in which these certain embodiments of the second and the third target reagent are included in the the plurality of target reagents, the candidate ABM may be characterized as specific for both the first and the second target antigen when, in addition to the second and/or additional barcoded nucleic acid molecules, both the third and fourth barcoded nucleic acid molecules are generated. In embodiments in which these certain embodiments of the second and the third target reagents are included in the the plurality of target reagents, the candidate ABM may be characterized as specific for the first target antigen when, in addition to the second and/or additional barcoded nucleic acid molecules, the third, but not the fourth, barcoded nucleic acid molecule is generated. In embodiments in which these certain embodiments of the second and the third target reagents are included in the the plurality of target reagents, the candidate ABM may be characterized as specific for the second target antigen when, in addition to the second and/or additional barcoded nucleic acid molecules, the fourth, but not the third, barcoded nucleic acid molecule is generated.

In any of the embodiments of the methods provided herein, the barcoded nucleic acid molecules generated may be sequenced, e.g., to determine a partition-specific barcode sequence, a reporter oligonucleotide-associated sequence (e.g., reporter barcode sequence) or a sequence encoding at least a portion of an ABM. Sequencing of any of the generated barcoded nucleic acid molecules may be performed by any of a variety of approaches, systems, or techniques, including next-generation sequencing (NGS) methods. Sequencing may be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, nanopore sequencing (Oxford Nanopore) and SMRT® sequencing.

Further, sequence analysis of the barcoded nucleic acid molecules may be direct or indirect. Thus, the sequence analysis can be performed on a barcoded nucleic acid molecule or it can be a molecule which is derived therefrom (e.g., a complement or amplicon thereof).

Other examples of methods for sequencing the barcoded nucleic acid molecules include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, whole transcriptome sequencing, and any combinations thereof.

It will be understood that in any of the methods provided herein, the plurality of target reagents is not necessarily limited to including the: (i) first target reagent, e.g., including the first target antigen and the second target antigen; or (ii) first target reagent, (e.g., including the first target antigen and the second target antigen), second target reagent, (e.g., including the first target antigen), and third target reagent, (e.g, including the second target antigen). Rather, the plurality of target reagents may further include one or more control reagent and/or one or more further target reagent.

A control reagent that may be included in the plurality of target reagents may include a control antigen. In some embodiments, the control antigen may be a protein or peptide antigen to which the plurality of cells expressing ABMs for inclusion in the reaction mixture would not expected to bind. By way of example, the control antigen may be a protein or peptide which a subject (e.g., a human subject from whom a plurality cells expressing ABMs would be obtained for use in the methods), would not be expected to have developed an immune response against. Such a control antigen may be a protein or peptide endogenous to and abundantly expressed in the subject, e.g., a serum albumin.

By way of other example, the control antigen may include an MHC molecule complexed with a control peptide fragment. In such examples, the control antigen may include an MHC molecule complexed with a peptide fragment of an abundantly expressed self-antigen (of a subject from whom the plurality of cells expressing ABMs had been obtained), a heteroclitic peptide, or a peptide to which the plurality cells expressing an ABM (obtained from a subject for inclusion in the reaction mixture) are naïve. Examples of abundantly expressed self-antigens of a subject (from whom the plurality of cells expressing ABMs may be obtained for use in the methods), peptide fragments of which may be complexed with an MHC molecule of the control antigen include a serum albumin, cardiolipin, keyhole limpet hemocyanin, flagellin, insulin. Examples of heteroclitic peptides that may be complexed with an MHC molecule of a control antigen generally include peptides having a valine, or leucine or other suitable residue at positions that anchor the peptide to the MHC molecule, e.g., position 2 and/or C-terminal residue, but alanine residues at the remaining amino acid positions (e.g., ALAAAAAAV, ATAAAAAAK, AYAAAAAAL, APAAAAAAV or RYAAAAALL). Examples of peptide fragments that may be complexed with an MHC molecule of a control antigen, where the fragments are of an antigen to which the plurality of cells expressing an ABM (obtained from a subject for inclusion in the reaction mixture) are naïve include a peptide or peptide fragment of an antigen of a virus, e.g. HIV (e.g., TPGPGVRYPL, SLYNTVATL, TPGPGVRYPL), if the subject from which the plurality of cells have been obtained, has not been exposed to the virus, e.g., HIV. Additional examples of peptide fragments that may be bound to an MHC molecule of a control antigen of the control reagent include peptide ASYAAAAV and vaccinia virus peptide TSYKFESV.

Any control reagent provided herein may include core support. The core support may be one or more of avidin, deglycosylated avidin, traptavidin, tamavidin, xenavidin, or bradavidin. In instances in which the core support includes one or more of avidin, deglycosylated avidin, traptavidin, tamavidin, xenavidin, or bradavidin, the control antigen may be biotinylated. In some instances in any of the methods provided herein, the control reagent may include the core support, e.g, avidin, deglycosylated avidin, traptavidin, tamavidin, xenavidin, or bradavidin, coupled to biotin, e.g, absent the control antigen.

Any control reagent provided herein may include a control reporter oligonucleotide and/or a fluorescent molecule. In instances in which the control reagent includes a control reporter oligonucleotide, the control oligonucleotide may include a control reporter barcode sequence. The control reporter barcode sequence may identify the control reagent. The control reporter oligonucleotide may further include, e.g., further to the control reporter barcode sequence, a capture handle sequence. The capture sequence of the control reporter oligonucleotide may be configured to couple to a capture sequence of a nucleic acid barcode molecule of the plurality of nucleic acid barcode molecules that may be in a provided partition. A control barcoded nucleic acid molecule may be generated in embodiments of the methods where the control reagent includes the control reporter oligonucleotide. A control barcoded nucleic acid molecule may include a sequence of the control reporter oligonucleotide or a reverse complement thereof and the partition-specific barcode sequence or reverse complement thereof. In instances in which the control reagent includes a fluorescent molecule, the fluorescent molecule may be a different fluorescent molecule, or a fluorescent molecule that emits a different fluorescent signal, relative to any first and/or additional fluorescent molecule that may be included in the first target reagent.

In instances in which the plurality of target reagents includes one or more further target reagents, the one or more further target reagents may include a further target antigen to which binding by an ABM is desirable, and that is different from the first target reagent's first and second target antigen. The further target antigen may be, as discussed earlier herein above, any antigen associated with an infectious agent, such as a viral, bacterial, parasitic, protozoal or prion agent, or a fragment of any such antigen complexed with an MHC molecule. The further target antigen may be any antigen associated with a tumor or a cancer, e.g., growth factor receptor or transcription factor, or a fragment of any such antigen complexed with an MHC molecule. Further, the further target antigen may be an antigen associated with a degenerative condition or disease, or may be a cytokine, a co-stimulatory molecule, a co-inhibitory molecule, a growth factor, or an ion channel, or it may be a fragment of any such antigen (e.g., associated with a degenerative condition or disease, or may be a cytokine, a co-stimulatory molecule, a co-inhibitory molecule, a growth factor, or an ion channel) complexed with an MHC molecule. In addition, the further target antigen may be or include a target lipid complexed with an MHC molecule or a target small molecule complexed with an MHC molecule.

The further target reagent, including the further target antigen, may also include a core support. The core support may be one or more of avidin, deglycosylated avidin, traptavidin, tamavidin, xenavidin, or bradavidin. In instances in which the core support includes one or more of avidin, deglycosylated avidin, traptavidin, tamavidin, xenavidin, or bradavidin, the further antigen may be biotinylated. The further target reagent may also, in addition to the further target antigen (and, optionally, the core support) include a further reporter oligonucleotide and/or a fluorescent molecule. In instances in which the further target reagent includes a further reporter oligonucleotide, the further oligonucleotide may include a further reporter barcode sequence. The further reporter barcode sequence may identify the further reagent. The further reporter oligonucleotide may additionally include, e.g., in addition to the further reporter barcode sequence, a capture handle sequence. The capture sequence of the further reporter oligonucleotide may be configured to couple to a capture sequence of a nucleic acid barcode molecule of the plurality of nucleic acid barcode molecules that may be in a provided partition. A further target barcoded nucleic acid molecule may be generated in embodiments of the methods where the further target reagent includes the further reporter oligonucleotide. A further target barcoded nucleic acid molecule may include a sequence of the further reporter oligonucleotide or a reverse complement thereof and the partition-specific barcode sequence or reverse complement thereof.

In instances in which the plurality of target reagents includes the further target reagent including a fluorescent molecule, (in combination with the first target antigen including the first and/or additional fluorescent molecule), the fluorescent molecule of the further target reagent may be a fluorescent molecule different from the first and the additional fluorescent molecule. In instances in which the plurality of target reagents includes the further target reagent including a fluorescent molecule, (in combination with the first target antigen including the first and/or additional fluorescent molecule and a control reagent including a control fluorescent molecule), the fluorescent molecule of the further target reagent may be a fluorescent molecule that is the same different from the first and the additional fluorescent molecule, and that is different from the control reagent's fluorescent molecule.

It will be be understood that in any of the methods provided herein, the plurality of target reagents is not necessarily limited to including a single further target reagent. Rather, the inclusion of a further target reagent in the the plurality of target reagents may refer to the inclusion of one, at least one, two, at least two, three, at least three, four, at least four, five, at least five, six, at least six, seven, at least seven, eight, at least eight, nine, at least nine, ten, at least ten, twenty, at least twenty, thirty, at least thirty, forty, at least forty, fifty, at least fifty, sixty, at least sixty, seventy, at least seventy, eighty, at least eighty, ninety, at least ninety, one hundred, at least one hundred, five hundred, or at least five hundred additional further target reagents in the plurality of target reagents.

In any of the methods described herein, it will be understood that the partitioning of the reaction mixture, or portion thereof, may partition more than one cell of the plurality of, e.g. B or T, cells expressing ABMs into more than one of a plurality of partitions. The partitioning of the reaction mixture may partition a first cell of the plurality of cells expressing ABMs into a first partition, it may further partition a second cell of the plurality of cells expressing ABMs into a second partition. Moreover, it may additionally partition a third cell of the plurality of cells expressing ABMs into a third partition, a fourth cell of the plurality of cells expressing ABMs into a fourth partition, up to hundreds, thousands, tens of thousands, hundreds of thousands, or millions of cells that are each partitioned into a separate, individual, partition. It should be understood that each and every partitioned cell need be bound to one or more in particular of the target reagents. However, at least one cell of the plurality of cells expressing ABMs partitioned into a partition will be bound to a target reagent. Further, it will be understood that the partitioning of the reaction mixture, or portion thereof, if it partitions an cell of a plurality of cells expressing ABMs, may partition a B cell in a first partition and a T cell in a second partition, e.g., not all partitions will necessarily include a B cell and not all partitions will necessarily include a T cell.

### Compositions

In another aspect, the disclosure provides for compositions. The compositions may be used to perform the methods provided herein, may be employed in the systems provided herein, or may be included in a kit. Accordingly, the compositions may be useful in the characterization of an ABM, e.g. TCR, BCR, Ab or antigen-binding fragment of an Ab.

The compositions of the disclosure may include a target reagent. In a first embodiment of the compositions of the disclosure, the target reagent may include a first target antigen, a second target antigen and a first reporter oligonucleotide. The first reporter oligonucleotide may include a first reporter barcode sequence. The first reporter barcode sequence may identify the target reagent.

In some of the first embodiments, the target reagent may further include an additional reporter oligonucleotide. In instances in which the target reagent further includes the additional reporter oligonucleotide, the first target antigen may be coupled to the first reporter oligonucleotide and the second target antigen may be coupled to the additional oligonucleotide. In embodiments in which the target reagent further includes the additional reporter oligonucleotide, the first reporter oligonucleotide (coupled to the first target antigen), may include a first reporter barcode sequence. The first reporter barcode sequence may identify the first target antigen. Further, in embodiments in which the target reagent further includes the additional reporter oligonucleotide, the additional reporter oligonucleotide (coupled to the second target antigen) may include an additional barcode sequence. The additional barcode sequence may identify the second target antigen.

In some of the first embodiments, the target reagent may further include a detectable label. The detectable label may be a fluorescent molecule or may be magnetic. In an example of such a first embodiment, the target reagent may include a first target antigen, a second target antigen, a first reporter oligonucleotide, and a first fluorescent molecule.

In yet other of the first embodiments, the target reagent may include a first target antigen, a second target antigen, a first reporter oligonucleotide, first fluorescent molecule, and an additional fluorescent molecule. In some of these first embodiments, the first target antigen may be coupled to the first fluorescent molecule and the second target antigen may be coupled to the additional fluorescent molecule. The first and the additional fluorescent molecule may emit first and second detectable signals. Example fluorescent molecules that may be included in the target reagent have been provided earlier herein, e.g., in the “METHODS OF THE DISCLOSURE.”

Example target reagents of these first embodiments of compositions of the disclosure include those which are comprised of a first target antigen, second target antigen, and: (i) a first reporter oligonucleotide; (ii) a first and an additional oligonucleotide; (iii) a first reporter oligonucleotide and a first fluorescent molecule; (iv) a first reporter oligonucleotide, a first fluorescent molecule and an additional fluorescent molecule; (v) a first reporter oligonucleotide, an additional reporter oligonucleotide and a first fluorescent molecule; or (vi) a first reporter oligonucleotide, an additional reporter oligonucleotide, a first fluorescent molecule and an additional fluorescent molecule. Configurations of these various first embodiments of the target reagent have been described in the “METHODS OF THE DISCLOSURE”, e.g., describing configurations of first target reagent herein.

The first embodiments of the compositions provided herein may include second and/or third target reagents, in addition to the target reagent. The second target reagent may include the first target antigen and a second reporter oligonucleotide. The second target oligonucleotide may include a second reporter barcode sequence, and the second reporter barcode sequence may identify the second target reagent (and the first target antigen of the second target reagent). The second target oligonucleotide may further include a capture handle sequence. The third target reagent may include the second target antigen and a third reporter oligonucleotide. The third target oligonucleotide may include a third reporter barcode sequence, and the third reporter barcode sequence may identify the third target reagent (and the second target antigen of the third target reagent). The third target oligonucleotide may further include capture handle sequence.

In a second embodiment of the compositions of the disclosure, the compositions may include a target reagent where the target reagent includes a first target antigen, a second target antigen and a first fluorescent molecule. In some of the second embodiments of the disclosure, the target reagent may further include an additional fluorescent molecule. In such embodiments, the first fluorescent molecule may be coupled to the first target antigen and the additional fluorescent molecule may be coupled to the second target antigen. The first fluorescent molecule and the additional fluorescent molecule may emit first and additional detectable signals.

In the second embodiment of the compositions of the disclosure, a second and/or third target reagent may be included with the target reagent (including the first target antigen, second target antigen, first fluorescent molecule and, optional, additional fluorescent molecule). In such second embodiments, the second target reagent may include the first target antigen and a second fluorescent molecule and the third target reagent may include the second target antigen and a third fluorescent molecule.

In any instance of the embodiments of the compositions of the disclosure, the first and the second target antigen of the target reagent may be coupled to a core support. The core support may include any of streptavidin, avidin, deglycosylated avidin, traptavidin, tamavidin, xenavidin, or bradavidin. In instances in which the core support includes any of streptavidin, avidin, deglycosylated avidin, traptavidin, tamavidin, xenavidin, or bradavidin, the first and the second target antigen of the target reagent may be biotinylated.

In any embodiments of the compositions of the disclosure that include the second target reagent and/or third target reagent, the second target reagent or the third target reagent may include a core support. In these embodiments, the core support may be any of streptavidin, avidin, deglycosylated avidin, traptavidin, tamavidin, xenavidin, or bradavidin. Further, in these embodiments, the first target antigen of the second target reagent and/or the second target antigen of the third target reagent may be biotinylated for coupling to their, respective, core support.

In any of the embodiments of the compositions of the disclosure, the first and/or second target antigen of the target reagent may be any target antigens to which identification and characterization of an ABM having binding specificity therefor is desirable. The first and/or second target antigen, as discussed earlier herein, may be any antigen or antigens associated with an infectious agent, such as a viral, bacterial, parasitic, protozoal or prion agent, or a fragment of any such antigen(s) bound to an MHC molecule. The first and/or second target antigen may be any antigen or antigens associated with a tumor or a cancer, e.g., growth factor receptor or transcription factor, or a fragment of any such antigen(s) complexed with an MHC molecule. Further, the first and/or second target antigen may be an antigen or antigens associated with a degenerative condition or disease, or may be one or more of a cytokine(s), co-stimulatory molecule(s), co-inhibitory molecule(s), growth factor(s), or ion channel(s), or a fragment or fragments of any such antigen(s) (e.g., associated with a degenerative condition or disease, or of a cytokine, co-stimulatory molecule, co-inhibitory molecule, growth factor, or ion channel) complexed with an MHC molecule. In addition, the first or second target antigen may be or include a target lipid bound to an MHC molecule or a target small molecule complexed with an MHC molecule. Further disclosure of first and second target antigens, including disclosure of MHC molecule alleles and selection of fragments of antigens for complexing with the MHC molecules, e.g., alleles, have been provided herein, e.g., in the “METHODS OF THE DISCLOSURE”.

In any of the embodiments of the compositions of the disclosure, the composition may further include a control reagent. The control reagent may include a control antigen and a control reporter oligonucleotide. In some instances, control antigen may be a protein or peptide antigen to which a plurality of cells expressing ABMs, for use with the composition, would not expected to bind. By way of example, the control antigen may be a protein or peptide for which a subject (e.g., a human subject from whom a plurality cells expressing ABMs would be obtained for use with the compositions), would not be expected to have developed an immune response against, e.g., a serum albumin. In other instances, the control reagent may include an MHC molecule bound to a control peptide fragment. In instances in which the control reagent includes an MHC molecule bound to a control peptide fragment, the control peptide fragment may be a peptide fragment of an abundantly expressed self-antigen (of a subject from whom a plurality of cells expressing ABMs will have been obtained for use with the composition), a heteroclitic peptide, or a peptide to which a plurality cells expressing an ABM (obtained from a subject for use with the composition) are naïve. Example of control peptide fragments for inclusion with the MHC molecule of such a control reagent have been described in the “METHODS OF THE DISCLOSURE”.

The control reporter oligonucleotide, further included with the control antigen of the control reagent, may include a control reporter barcode sequence. The control reporter barcode sequence may identify the control reagent. The control reporter oligonucleotide may further include, e.g., further to the control reporter barcode sequence, a capture handle sequence.

In some instances, the control reagent may further include a detectable label, e.g., fluorescent or magnetic molecule.

In some instances, the control reagent may include a core support. The core support may be one or more of avidin, deglycosylated avidin, traptavidin, tamavidin, xenavidin, or bradavidin. In instances in which the core support includes one or more of avidin, deglycosylated avidin, traptavidin, tamavidin, xenavidin, or bradavidin, the control antigen may be biotinylated.

In any instances of compositions provided herein, the control reagent that may be included in the composition may include the core support, e.g, avidin, deglycosylated avidin, traptavidin, tamavidin, xenavidin, or bradavidin, coupled to biotin, e.g, absent the control antigen.

In any of the embodiments of the compositions of the disclosure, the composition may further include a further target reagent. The further target reagent may include a further target antigen to which binding by an ABM is desirable, and that is different from the first target reagent's first and second target antigen. Examples of further target antigens have been discussed in the “METHODS OF THE DISCLOSURE”.

The further target reagent, including the further target antigen, may also include a core support. The core support may include avidin, deglycosylated avidin, traptavidin, tamavidin, xenavidin, or bradavidin. In instances in which the core support includes avidin, deglycosylated avidin, traptavidin, tamavidin, xenavidin, or bradavidin, the further antigen may be biotinylated. The further target reagent may also, in addition to the further target antigen (and, optionally, the core support) include a further reporter oligonucleotide and/or a fluorescent molecule. In instances in which the further target reagent includes a further reporter oligonucleotide, the further oligonucleotide may include a further reporter barcode sequence. The further reporter barcode sequence may identify the further reagent. The further reporter oligonucleotide may additionally include, e.g., in addition to the further reporter barcode sequence, a capture handle sequence.

It will be be understood that in any of the compositions provided herein, the inclusion of the further target reagent in the composition may refer to the inclusion of one, at least one, two, at least two, three, at least three, four, at least four, five, at least five, six, at least six, seven, at least seven, eight, at least eight, nine, at least nine, ten, at least ten, twenty, at least twenty, thirty, at least thirty, forty, at least forty, fifty, at least fifty, sixty, at least sixty, seventy, at least seventy, eighty, at least eighty, ninety, at least ninety, one hundred, at least one hundred, five hundred, or at least five hundred further target reagents in the composition.

Any of the compositions provided herein above, e.g., compositions that include the target reagent and that may further include the second and/or third and/or one or more control, and/or one or more further target reagents, may be included in a kit. Such kits may include instructions for use. The instructions for use may be provided in any format, e.g., they may be recorded on a suitable recording medium. For example, the instructions may be printed on a substrate, such as paper or plastic, etc. In another example, the instructions may be present as an electronic storage data file present on a suitable computer readable storage medium, e.g. CD-ROM, diskette, flash drive, etc. In some instances, the instructions are not physically present in the kit, but means for obtaining the instructions from a remote source (e.g., via the internet), are provided. An example of this embodiment is a kit that includes a web address where the instructions can be viewed and/or from which the instructions can be downloaded.

Any of the compositions provided herein, e.g., including a target reagent, may further include a cell. The cell may be any cell that expresses an ABM. The cell may be a B cell, e.g., cell of B cell lineage such as a memory B cell which expresses an antibody as a cell surface receptor or a plasma cell that secretes an antibody. The cell may be a T cell. In embodiments in which the composition includes a cell expressing an ABM, e.g., B or T cell, the cell may be bound to the target reagent or may have been bound to the target reagent. By way of example, the composition may include a T cell bound to, or that had bound to, the target reagent. By way of other example, the composition may include B cell bound to, or that had bound to, the target reagent.

Any of the compositions provided herein may be in a partition. Partitions are discussed extensively herein, and include wells, microwell, and droplets. In instances in which the compositions are in a partition the composition may further include a plurality of nucleic acid molecules.

Further disclosure related to these reagents can be found in the “Further Disclosure-Partitions, Partitioning, Reagents and Processing” section, immediately below.

### Systems

In an aspect, the disclosure provides for a system. The system may be useful to implement the methods provided herein, e.g., methods that characterize an ABM, e.g., TCR, BCR, Ab or antigen-binding fragment of an Ab. The system may include a target reagent. In one embodiment, the system may include a target reagent where the target reagent includes a first target antigen, a second target antigen and a first reporter oligonucleotide. The first reporter oligonucleotide may include a first reporter barcode sequence. The first reporter barcode sequence may identify the target reagent.

In some of the first embodiments, the target reagent may further include an additional reporter oligonucleotide. In instances in which the target reagent further includes the additional reporter oligonucleotide, the first target antigen may be coupled to the first reporter oligonucleotide and the second target antigen may be coupled to the additional oligonucleotide. In embodiments in which the target reagent further includes the additional reporter oligonucleotide, the first reporter oligonucleotide (coupled to the first target antigen), may include a first reporter barcode sequence and the first reporter barcode sequence may identify the first target antigen. Further, in embodiments in which the target reagent further includes the additional reporter oligonucleotide, the additional reporter oligonucleotide (coupled to the second target antigen) may include an additional barcode sequence and the additional barcode sequence may identify the second target antigen.

In some of the first embodiments of the systems, the target reagent may further include a detectable label. The detectable label may be a fluorescent molecule or may be magnetic. Accordingly, in some of the first embodiments of the systems, the target reagent may include a first target antigen, a second target antigen, a first reporter oligonucleotide, and a first fluorescent molecule. In other of the first embodiments of the systems, the target reagent may include a first target antigen, a second target antigen, a first reporter oligonucleotide, and first fluorescent molecule and second fluorescent molecule. In some other of these first embodiments of the systems, the first target antigen may be coupled to the first fluorescent molecule and the second target antigen may be coupled to the second fluorescent molecule. The first and the second fluorescent molecule may emit first and second detectable signals. Example fluorescent molecules that may be included in the target reagent have been provided earlier herein, e.g., in the “METHODS OF THE DISCLOSURE.”

Examples of the target reagent in first embodiments of systems of the disclosure may include a first target antigen, second target antigen, and: (i) a first reporter oligonucleotide; (ii) a first and a further oligonucleotide; (iii) a first reporter oligonucleotide and a first fluorescent molecule; (iv) a first reporter oligonucleotide, a first fluorescent molecule and an second fluorescent molecule; (v) a first reporter oligonucleotide, a second reporter oligonucleotide and a first fluorescent molecule; or (vi) a first reporter oligonucleotide, a second reporter oligonucleotide, a first fluorescent molecule and an second fluorescent molecule. Configurations of these various first embodiments of the target reagent have been described in the “METHODS OF THE DISCLOSURE”, e.g., describing configurations of first target reagent.

The first embodiments of the systems provided herein may include second and/or third target reagents, in addition to the target reagent (including the first and the second target antigen). The second target reagent may include the first target antigen and a second reporter oligonucleotide. The second target oligonucleotide may include a second reporter barcode sequence, and the second reporter barcode sequence may identify the second target reagent (and the first target antigen of the second target reagent). The second target oligonucleotide may further include a capture handle sequence. The third target reagent may include the second target antigen and a third reporter oligonucleotide. The third target oligonucleotide may include a third reporter barcode sequence, and the third reporter barcode sequence may identify the third target reagent (and the second target antigen of the third target reagent). The third target oligonucleotide may further include capture handle sequence.

In a second embodiment of the systems of the disclosure, the systems may include a target reagent where the target reagent includes a first target antigen, a second target antigen and a first fluorescent molecule. In some of the second embodiments of the systems of the disclosure, the target reagent may further include an additional fluorescent molecule. In such embodiments, the first fluorescent molecule may be coupled to the first target antigen and the additional fluorescent molecule may be coupled to the second target antigen. The first fluorescent molecule and the additional fluorescent molecule may emit first and additional detectable signals.

In the second embodiment of the systems of the disclosure, a second and/or third target reagent may be included with the target reagent (including the first target antigen, second target antigen, first fluorescent molecule and, optional additional fluorescent molecule). In such second embodiments, the second target reagent may include the first target antigen and a second fluorescent molecule and the third target reagent may include the second target antigen and a third fluorescent molecule.

In any of the embodiments of the systems of the disclosure, the target reagent, the second target reagent and/or the third target reagent may include a core support. The core support may include any of streptavidin, avidin, deglycosylated avidin, traptavidin, tamavidin, xenavidin, or bradavidin. In instances in which the core support includes any of streptavidin, avidin, deglycosylated avidin, traptavidin, tamavidin, xenavidin, or bradavidin, the first and the second target antigen of the target reagent, second target reagent and/or third target reagent may be biotinylated.

In any of the embodiments of the systems of the disclosure, the first and/or second target antigen of the target reagent may be any target antigens to which identification and characterization of an ABM having binding specificity therefor is desirable. The first and/or second target antigen, may be antigen or antigens as discussed earlier herein. Sec, e.g. “METHODS OF THE DISCLOSURE”.

In any of the embodiments of the systems of the disclosure, the system may further include a control reagent. The control reagent may include a control antigen and a control reporter oligonucleotide. In some instances, the control antigen may be a protein or peptide antigen to which a plurality of cells expressing ABMs, for use in the system, would not expected to bind, e.g, a serum albumin. In other instances, the control reagent may include an MHC molecule bound to a control peptide fragment. Example of control peptide fragments for inclusion with the MHC molecule of such a control reagent have been described in the “METHODS OF THE DISCLOSURE”. The control reporter oligonucleotide, further included with the control antigen of the control reagent, may include a control reporter barcode sequence. The control reporter barcode sequence may identify the control reagent. The control reporter oligonucleotide may further include, e.g., further to the control reporter barcode sequence, a capture handle sequence.

In some instances, the control reagent may further include, further to the control antigen and control reporter oligonucleotide, a detectable label. The detectable label may be a fluorescent or magnetic molecule. In some instances, the control reagent may further include a core support. The core support may be one or more of avidin, deglycosylated avidin, traptavidin, tamavidin, xenavidin, or bradavidin. In instances in which the core support includes one or more of avidin, deglycosylated avidin, traptavidin, tamavidin, xenavidin, or bradavidin, the control antigen may be biotinylated. In any instances of compositions provided herein, the control reagent that may be included in the composition may include the core support, e.g, avidin, deglycosylated avidin, traptavidin, tamavidin, xenavidin, or bradavidin, coupled to biotin, e.g, absent the control antigen.

In any of the embodiments of the systems of the disclosure, the system may further include a further target reagent. The further target reagent may include a further target antigen to which binding by an ABM is desirable, and that is different from the first target reagent's first and second target antigen. The further target antigen may be any further target antigen provided earlier in the “METHODS OF THE DISCLOSURE”.

The further target reagent, including the further target antigen, may also include a core support as described herein. The further target reagent may also, in addition to the further target antigen (and, optionally, the core support) include a further reporter oligonucleotide and/or a fluorescent molecule. In instances in which the further target reagent includes a further reporter oligonucleotide, the further oligonucleotide may include a further reporter barcode sequence. The further reporter barcode sequence may identify the further reagent. The further reporter oligonucleotide may additionally include, e.g., in addition to the further reporter barcode sequence, a capture handle sequence.

Any embodiments of the systems provided herein may further include a plurality of nucleic acid barcode molecules. A nucleic acid barcode molecule of the plurality may include a partition-specific barcode sequence. In addition to the partition-specific barcode sequence, it may include a capture sequence. Nucleic acid barcode molecules of the plurality of nucleic acid barcode molecules may further include functional sequences as disclosed herein.

In any embodiment of the systems provided herein, the system may further include a partitioning system, which may be a microfluidic device. Microfluidic devices, channel structures and networks are discussed extensively herein at, for example, the section entitled “MICROFLUIDIC SYSTEMS.”

In any embodiments of the systems provided herein, the system may further include reagents for generating a first of a plurality of barcoded nucleic acid molecules formed by complementary base pairing of: (a) the capture sequence of the plurality of nucleic acid barcode molecules and (b) a sequence of an mRNA or DNA analyte encoding at least a portion of an ABM, e.g., TCR, BCR, Ab or antigen-binding fragment of the Ab.

The systems provided herein may further include a sequencer or sequencing system for sequencing the first of the plurality of barcoded nucleic acid molecules, an analysis engine, a network, an analysis engine and/or a network.

### Further Disclosure—Partitions, Partitioning, Reagents and Processing

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. A partition can be a volume or subvolume wherein diffusion of contents beyond the volume or sub-volume is inhibited. For example, the partitions can include a porous matrix that is capable of entraining and/or retaining materials within its matrix. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead.

Unique identifiers, such as barcodes, may be injected into the droplets previous to, subsequent to, or concurrently with droplet generation, such as via a bead, as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

The partitions described herein may comprise small volumes, for example, less than about 10 microliters (μL), 5 μL, 1 μL, 10 nanoliters (nL), 5 nL, 1 nL, 900 picoliters (pL), 800 μL, 700 pL, 600 μL, 500 pL, 400 pL, 300 μL, 200 pL, 100 pL, 50 μL, 20 pL, 10 μL, 1 pL, 500 nanoliters (nL), 100 nL, 50 nL, or less.

For example, in the case of droplet based partitions, the droplets may have overall volumes that are less than about 1000 μL, 900 pL, 800 μL, 700 pL, 600 μL, 500 pL, 400 pL, 300 μL, 200 pL, 100 pL, 50 μL, 20 pL, 10 μL, 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Application No. PCT/US20/17785, which is entirely incorporated herein by reference for all purposes.

### Microfluidic Systems

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

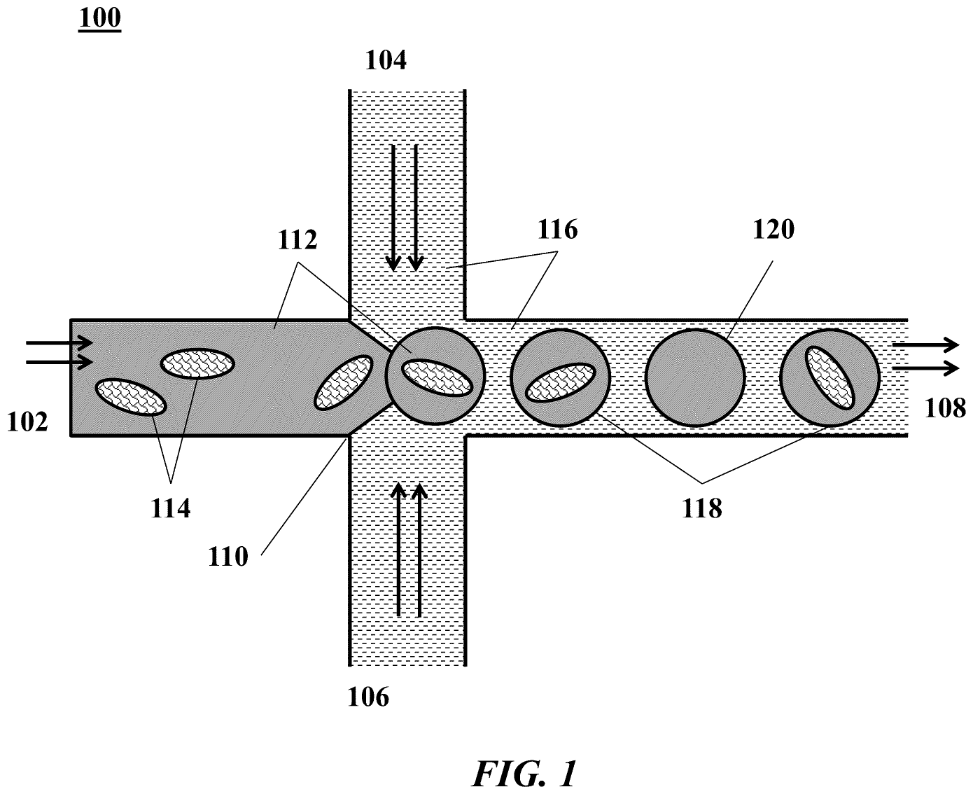

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as beads (e.g., gel beads) carrying nucleic acid barcode molecules (e.g., oligonucleotides).

In some examples, a partition of the plurality of partitions may comprise a single biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety.

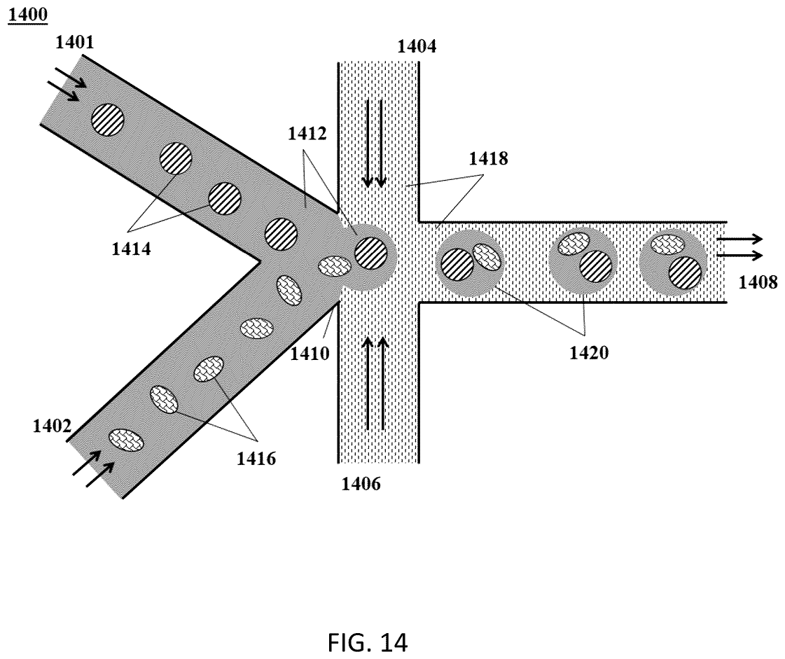

FIG. 14 shows an example of a microfluidic channel structure 1400 for delivering barcode carrying beads to droplets. The channel structure 1400 can include channel segments 1401, 1402, 1404, 1406 and 1408 communicating at a channel junction 1410. In operation, the channel segment 1401 may transport an aqueous fluid 1412 that includes a plurality of beads 1414 (e.g., with nucleic acid molecules, e.g., nucleic acid barcode molecules or barcoded oligonucleotides, molecular tags) along the channel segment 1401 into junction 1410. The plurality of beads 1414 may be sourced from a suspension of beads. For example, the channel segment 1401 may be connected to a reservoir comprising an aqueous suspension of beads 1414. The channel segment 1402 may transport the aqueous fluid 1412 that includes a plurality of biological particles 1416 along the channel segment 1402 into junction 1410. The plurality of biological particles 1416 may be sourced from a suspension of biological particles. For example, the channel segment 1402 may be connected to a reservoir comprising an aqueous suspension of biological particles 1416. In some instances, the aqueous fluid 1412 in either the first channel segment 1401 or the second channel segment 1402, or in both segments, can include one or more reagents, as further described below. A second fluid 1418 that is immiscible with the aqueous fluid 1412 (e.g., oil) can be delivered to the junction 1410 from each of channel segments 1404 and 1406. Upon meeting of the aqueous fluid 1412 from each of channel segments 1401 and 1402 and the second fluid 1418 from each of channel segments 1404 and 1406 at the channel junction 1410, the aqueous fluid 1412 can be partitioned as discrete droplets 1420 in the second fluid 1418 and flow away from the junction 1410 along channel segment 1408. The channel segment 1408 may deliver the discrete droplets to an outlet reservoir fluidly coupled to the channel segment 1408, where they may be harvested. As an alternative, the channel segments 1401 and 1402 may meet at another junction upstream of the junction 1410. At such junction, beads and biological particles may form a mixture that is directed along another channel to the junction 1410 to yield droplets 1420. The mixture may provide the beads and biological particles in an alternating fashion, such that, for example, a droplet comprises a single bead and a single biological particle.

### Controlled Partitioning

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

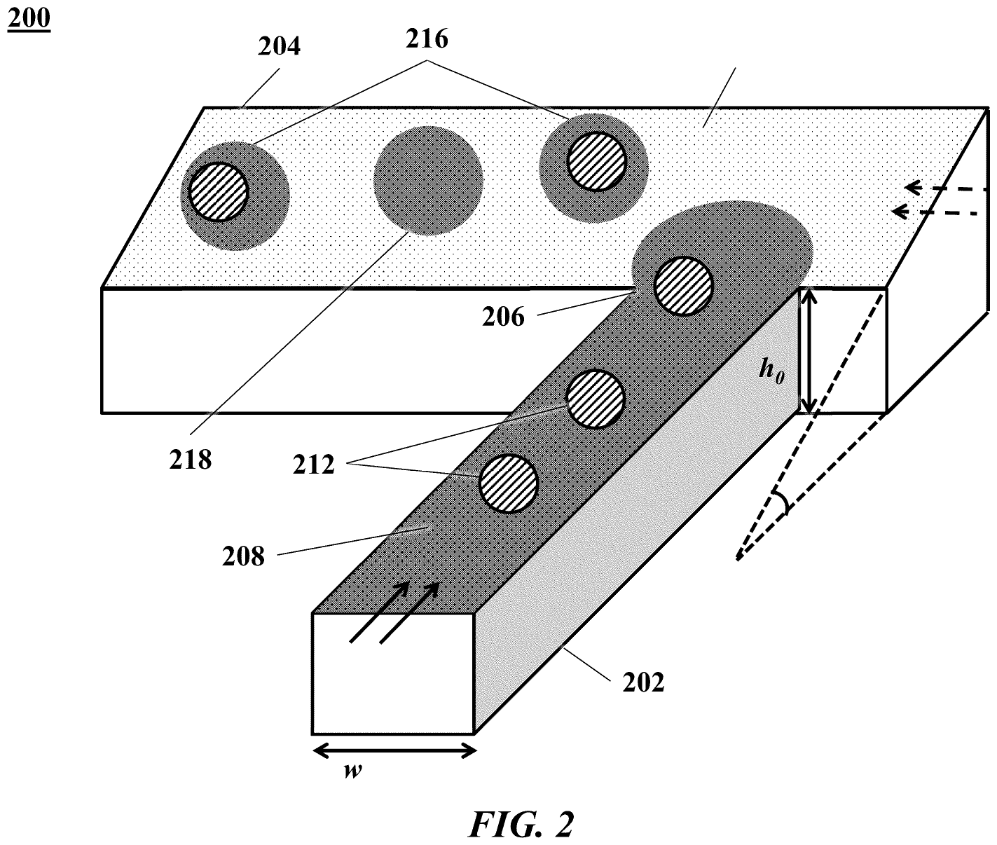

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 may be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, ho, a, etc.) of the channel structure 200. A plurality of droplets can be collected in the reservoir 204 by continuously injecting the aqueous fluid 208 from the channel segment 202 through the junction 206.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can comprise biological particles. In some instances, the aqueous fluid 208 can have a substantially uniform concentration or frequency of biological particles. As with the beads, the biological particles can be introduced into the channel segment 202 from a separate channel. The frequency or concentration of the biological particles in the aqueous fluid 208 in the channel segment 202 may be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads may be upstream or downstream of the second separate channel introducing the biological particles.

The second fluid 210 can comprise an oil, such as a fluorinated oil, that includes a fluorosurfactant for stabilizing the resulting droplets, for example, inhibiting subsequent coalescence of the resulting droplets.

In some instances, the second fluid 210 may not be subjected to and/or directed to any flow in or out of the reservoir 204. For example, the second fluid 210 may be substantially stationary in the reservoir 204. In some instances, the second fluid 210 may be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 may be subjected and/or directed to flow in or out of the reservoir 204. For example, the reservoir 204 can be a channel directing the second fluid 210 from upstream to downstream, transporting the generated droplets.

Systems and methods for controlled partitioning are described further in PCT/US2018/047551, which is hereby incorporated by reference in its entirety.

### Cell Beads

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example, after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents).

Suitable polymers or gels may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. The conditions sufficient to polymerize or gel the precursors may comprise any conditions sufficient to polymerize or gel the precursors. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), electromagnetic radiation, mechanical stimuli, or any combination thereof.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and PCT/US2018/016019, which are hereby incorporated by reference in their entirety. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles can be selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Exemplary stimuli suitable for degradation of the bead are described in U.S. Patent Application Publication No. 2014/0378345, which is entirely incorporated herein by reference for all purposes.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. The polymer or gel, e.g., polymer gel matrix, hydrogel or hydrogel matrix, may be functionalized to couple or link to a plurality of capture agents. The plurality of capture agents may, e.g., covalently or non-covalently, couple or link to the backbone of the polymer. Sec, e.g., U.S. Pat. No. 10,590,244, which is incorporated by reference in its entirety, for exemplary cell bead functionalization strategies. In an embodiment, a first capture agent of a plurality of capture agents may be a polypeptide or aptamer that (i) couples or links to the backbone of the polymer, and (ii) binds a specific analyte (e.g., antibody or antigen-binding fragment thereof) secreted by the cell, e.g., B cell. By way of example, a first capture agent of a plurality of capture agents may be a polypeptide, e.g., antibody, or aptamer that couples/links to the backbone of the polymer and binds to a secreted antibody, e.g., at its Fc region. It will be understood that, in some embodiments, the first capture agent of the plurality of capture agents may, rather than couple/link to the backbone of the polymer of the gel matrix, embed in/couple to the cell membrane. In these embodiments, the first capture agent, e.g., polypeptide or aptamer, may (i) embed in the membrane of the cell and/or bind to a cell surface protein and (ii) bind the specific analyte, e.g., antibody or antigen-binding fragment thereof, thereby tethering the secreted analyte, e.g., antibody, to the cell.

The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively or in addition, encapsulated biological particles may be readily deposited into other partitions (e.g., droplets) as described above.

### Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus. In some cases, nucleic acid barcode molecules are not released or not releaseable from the bead.

The solid support may be a bead. A solid support, e.g., a bead, may be porous, non-porous, hollow, solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In some cases, nucleic acid barcode molecules can be initially associated with the bead and then released from the bead. Release of the nucleic acid barcode molecules can be passive (e.g., by diffusion out of the bead). In addition or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in in US. Patent Publication Nos. 2019/0367997 and 2019/0064173, and International Application No. PCT/US20/17785, each of which is herein entirely incorporated by reference for all purposes.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Sec, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid barcode molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules are covalently linked to the bead via a linker. In some cases, the linker is a degradable linker. In some cases, the linker comprises a labile bond configured to release said nucleic acid barcode molecule of said plurality of nucleic acid barcode molecules. In some cases, the labile bond comprises a disulfide linkage.

Activation of disulfide linkages within a bead can be controlled such that only a small number of disulfide linkages are activated. Methods of controlling activation of disulfide linkages within a bead are described in PCT/US2014/044398, which is hereby incorporated by reference in its entirety.

In some cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is hereby incorporated by reference in its entirety. A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Scc, e.g., PCT/US2014/044398, which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively or in addition, species may be partitioned in a partition (e.g., droplet) during or subsequent to partition formation. Such species may include, without limitation, the abovementioned species that may also be encapsulated in a bead.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

### Nucleic Acid Barcode Molecules

A nucleic acid barcode molecule may contain one or more barcode sequences. A plurality of nucleic acid barcode molecules may be coupled to a bead. The one or more barcode sequences may include sequences that are the same for all nucleic acid molecules coupled to a given bead and/or sequences that are different across all nucleic acid molecules coupled to the given bead. The nucleic acid molecule may be incorporated into the bead.

Nucleic acid barcode molecules can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In some cases, the nucleic acid barcode molecule can further comprise a unique molecular identifier (UMI). In some cases, the nucleic acid barcode molecule can comprise one or more functional sequences, for example, for attachment to a sequencing flow cell, such as, for example, a P5 sequence (or a portion thereof) for Illumina® sequencing. In some cases, the nucleic acid barcode molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina sequencing. In some cases, the nucleic acid molecule can comprise an R1 primer sequence for Illumina sequencing. In some cases, the nucleic acid molecule can comprise an R2 primer sequence for Illumina sequencing. In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

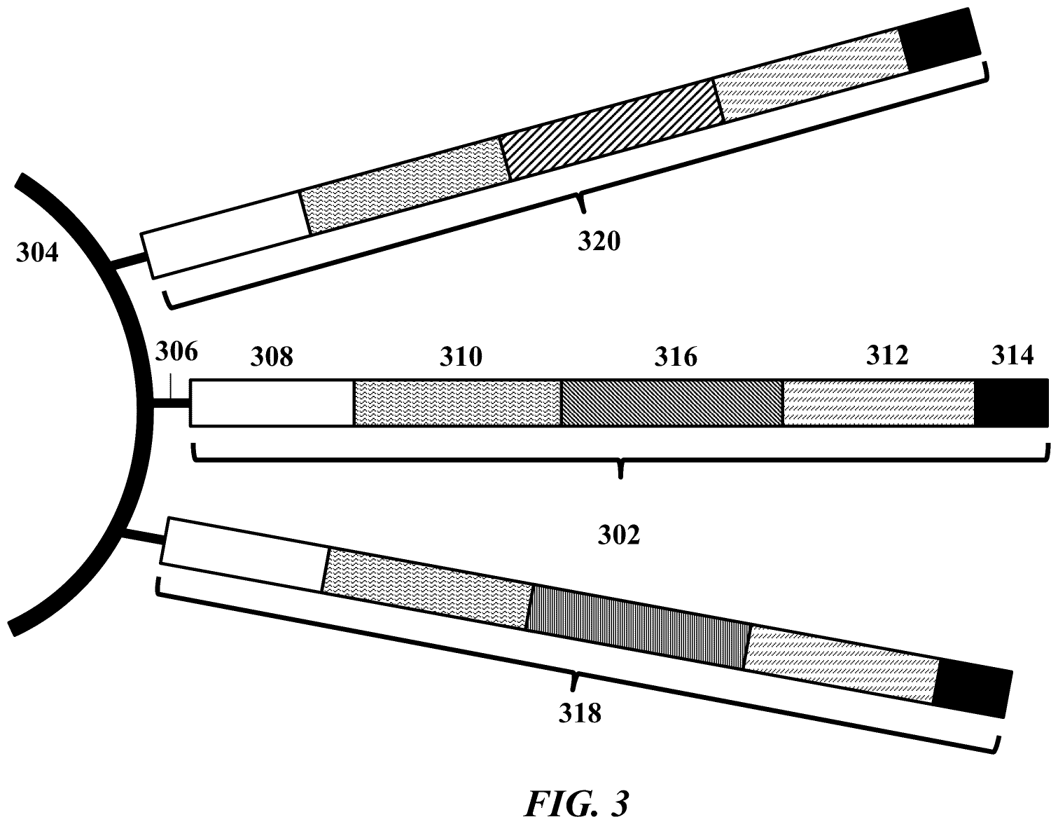

FIG. 3 illustrates an example of a barcode carrying bead. A nucleic acid barcode molecule 302 can be coupled to a bead 304 by a releasable linkage 306, such as, for example, a disulfide linker. The same bead 304 may be coupled (e.g., via releasable linkage) to one or more other nucleic acid barcode molecules 318, 320. The nucleic acid barcode molecule 302 may be or comprise a barcode. As noted elsewhere herein, the structure of the barcode may comprise a number of sequence elements. The nucleic acid barcode molecule 302 may comprise a functional sequence 308 that may be used in subsequent processing. For example, the functional sequence 308 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The nucleic acid barcode molecule 302 may comprise a barcode sequence 310 for use in barcoding the sample (e.g., DNA, RNA, protein, etc.). In some cases, the barcode sequence 310 can be bead-specific such that the barcode sequence 310 is common to all nucleic acid barcode molecules (e.g., including nucleic acid barcode molecule 302) coupled to the same bead 304. Alternatively or in addition, the barcode sequence 310 can be partition-specific such that the barcode sequence 310 is common to all nucleic acid barcode molecules coupled to one or more beads that are partitioned into the same partition. The nucleic acid barcode molecule 302 may comprise sequence 312 complementary to an analyte of interest, e.g., a priming sequence. Sequence 312 can be a poly-T sequence complementary to a poly-A tail of an mRNA analyte, a targeted priming sequence, and/or a random priming sequence. The nucleic acid barcode molecule 302 may comprise an anchoring sequence 314 to ensure that the specific priming sequence 312 hybridizes at the sequence end (e.g., of the mRNA). For example, the anchoring sequence 314 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid barcode molecule 302 may comprise a unique molecular identifying sequence 316 (e.g., unique molecular identifier (UMI)). In some cases, the unique molecular identifying sequence 316 may comprise from about 5 to about 8 nucleotides. Alternatively, the unique molecular identifying sequence 316 may compress less than about 5 or more than about 8 nucleotides. The unique molecular identifying sequence 316 may be a unique sequence that varies across individual nucleic acid barcode molecules (e.g., 302, 318, 320, etc.) coupled to a single bead (e.g., bead 304). In some cases, the unique molecular identifying sequence 316 may be a random sequence (e.g., such as a random N-mer sequence). For example, the UMI may provide a unique identifier of the starting analyte (e.g., mRNA) molecule that was captured, in order to allow quantitation of the number of original expressed RNA molecules. As will be appreciated, although FIG. 3 shows three nucleic acid barcode molecules 302, 318, 320 coupled to the surface of the bead 304, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both (i) common sequence segments or relatively common sequence segments (e.g., 308, 310, 312, etc.) and (ii) variable or unique sequence segments (e.g., 316) between different individual nucleic acid barcode molecules coupled to the same bead.

In operation, a biological particle (e.g., cell, DNA, RNA, etc.) can be co-partitioned along with a barcode bearing bead 304. The nucleic acid barcode molecules 302, 318, 320 can be released from the bead 304 in the partition. By way of example, in the context of analyzing sample RNA, the poly-T segment (e.g., 312) of one of the released nucleic acid barcode molecules (e.g., 302) can hybridize to the poly-A tail of a mRNA molecule. Reverse transcription may result in a cDNA transcript of the mRNA, but which transcript includes each of the sequence segments 308, 310, 316 of the nucleic acid barcode molecule 302. Because the nucleic acid barcode molecule 302 comprises an anchoring sequence 314, it will more likely hybridize to and prime reverse transcription at the sequence end of the poly-A tail of the mRNA. cDNA transcripts of the individual mRNA molecules from any given partition may include a common barcode sequence segment 310. However, the transcripts made from the different mRNA molecules within a given partition may vary at the unique molecular identifying sequence 312 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions may be collected, pooled, and subjected to further processing (e.g., reverse transcription, adapter attachment, amplification, clean up, sequencing). In other instances, one or more of the processing methods, e.g., reverse transcription, may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, an biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

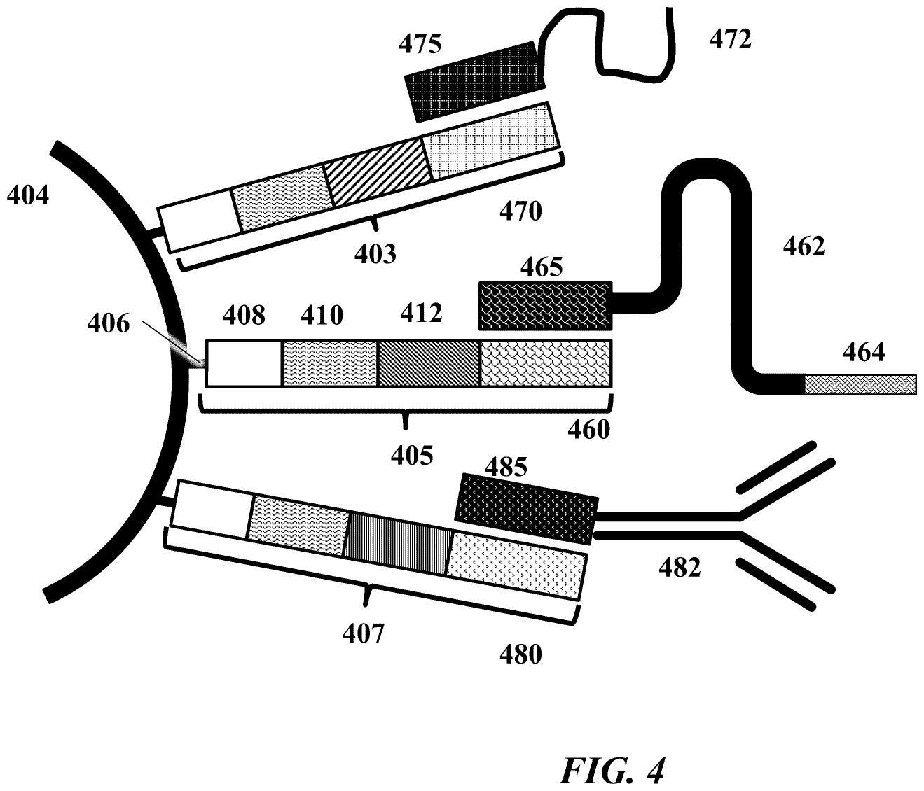

FIG. 4 illustrates another example of a barcode carrying bead. A nucleic acid barcode molecule 405, such as an oligonucleotide, can be coupled to a bead 404 by a releasable linkage 406, such as, for example, a disulfide linker. The nucleic acid barcode molecule 405 may comprise a first capture sequence 460. The same bead 404 may be coupled (e.g., via releasable linkage) to one or more other nucleic acid molecules 403, 407 comprising other capture sequences. The nucleic acid barcode molecule 405 may be or comprise a barcode. As noted elsewhere herein, the structure of the barcode may comprise a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 may be configured to attach to a corresponding capture sequence 465. In some instances, the corresponding capture sequence 465 may be coupled to another molecule that may be an analyte or an intermediary carrier. For example, as illustrated in FIG. 4, the corresponding capture sequence 465 is coupled to a guide RNA molecule 462 comprising a target sequence 464, wherein the target sequence 464 is configured to attach to the analyte. Another oligonucleotide molecule 407 attached to the bead 404 comprises a second capture sequence 480 which is configured to attach to a second corresponding capture sequence 485. As illustrated in FIG. 4, the second corresponding capture sequence 485 is coupled to an antibody 482. In some cases, the antibody 482 may have binding specificity to an analyte (e.g., surface protein). Alternatively, the antibody 482 may not have binding specificity. Another oligonucleotide molecule 403 attached to the bead 404 comprises a third capture sequence 470 which is configured to attach to a third corresponding capture sequence 475. As illustrated in FIG. 4, the third corresponding capture sequence 475 is coupled to a molecule 472. The molecule 472 may or may not be configured to target an analyte. The other oligonucleotide molecules 403, 407 may comprise the other sequences (e.g., functional sequence, barcode sequence, UMI, etc.) described with respect to oligonucleotide molecule 405. While a single oligonucleotide molecule comprising each capture sequence is illustrated in FIG. 4, it will be appreciated that, for each capture sequence, the bead may comprise a set of one or more oligonucleotide molecules each comprising the capture sequence. For example, the bead may comprise any number of sets of one or more different capture sequences. Alternatively or in addition, the bead 404 may comprise other capture sequences. Alternatively or in addition, the bead 404 may comprise fewer types of capture sequences (e.g., two capture sequences). Alternatively or in addition, the bead 404 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads are provided that each include large numbers of the above described nucleic acid barcode molecules releasably attached to the beads, where all of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in some cases, a chemical stimulus may be co-partitioned along with an encapsulated biological particle to allow for the degradation of the bead and release of the cell or its contents into the larger partition. In some cases, this stimulus may be the same as the stimulus described elsewhere herein for release of nucleic acid molecules (e.g., oligonucleotides) from their respective bead. In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particle, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some cases, template switching can be used to increase the length of a cDNA. In some cases, template switching can be used to append a predefined nucleic acid sequence to the cDNA. Template switching is further described in PCT/US2017/068320, which is hereby incorporated by reference in its entirety. Template switching oligonucleotides may comprise a hybridization region and a template region. Template switching oligonucleotides are further described in PCT/US2017/068320, which is hereby incorporated by reference in its entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In some aspects, this is performed by co-partitioning the individual biological particle or groups of biological particles with the unique identifiers, such as described above (with reference to FIG. 1 or 2).

In some cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

In some embodiments, following the generation of barcoded nucleic acid molecules according to methods disclosed herein, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations.

### Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 L, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well may comprise free reagents and/or reagents encapsulated in, or otherwise coupled to or associated with, beads, or droplets.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

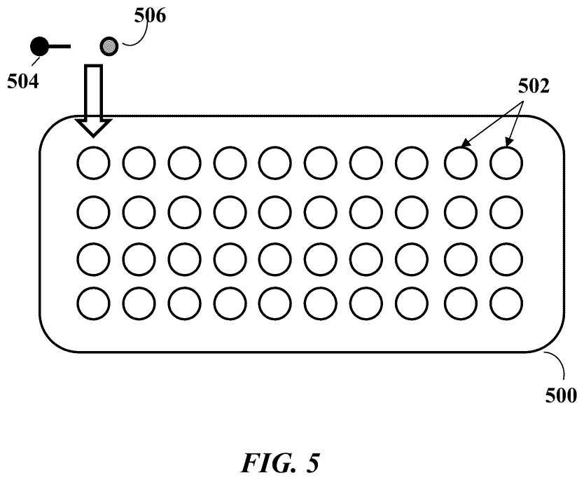

FIG. 5 schematically illustrates an example of a microwell array. The array can be contained within a substrate 500. The substrate 500 comprises a plurality of wells 502. The wells 502 may be of any size or shape, and the spacing between the wells, the number of wells per substrate, as well as the density of the wells on the substrate 500 can be modified, depending on the particular application. In one such example application, a sample molecule 506, which may comprise a cell or cellular components (e.g., nucleic acid molecules) is co-partitioned with a bead 504, which may comprise a nucleic acid barcode molecule coupled thereto. The wells 502 may be loaded using gravity or other loading technique (e.g., centrifugation, liquid handler, acoustic loading, optoelectronic, etc.). In some instances, at least one of the wells 502 contains a single sample molecule 506 (e.g., cell) and a single bead 504.

Reagents may be loaded into a well either sequentially or concurrently. In some cases, reagents are introduced to the device either before or after a particular operation. In some cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead, or droplet. These beads, or droplets may be loaded into a partition (e.g., a microwell) before, after, or concurrently with the loading of a cell, such that each cell is contacted with a different bead, or droplet. This technique may be used to attach a unique nucleic acid barcode molecule to nucleic acid molecules obtained from each cell. Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to: dynamic live-cell tracking, cell-cell interactions (when two or more cells are co-partitioned), cell proliferation, etc. Alternatively or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

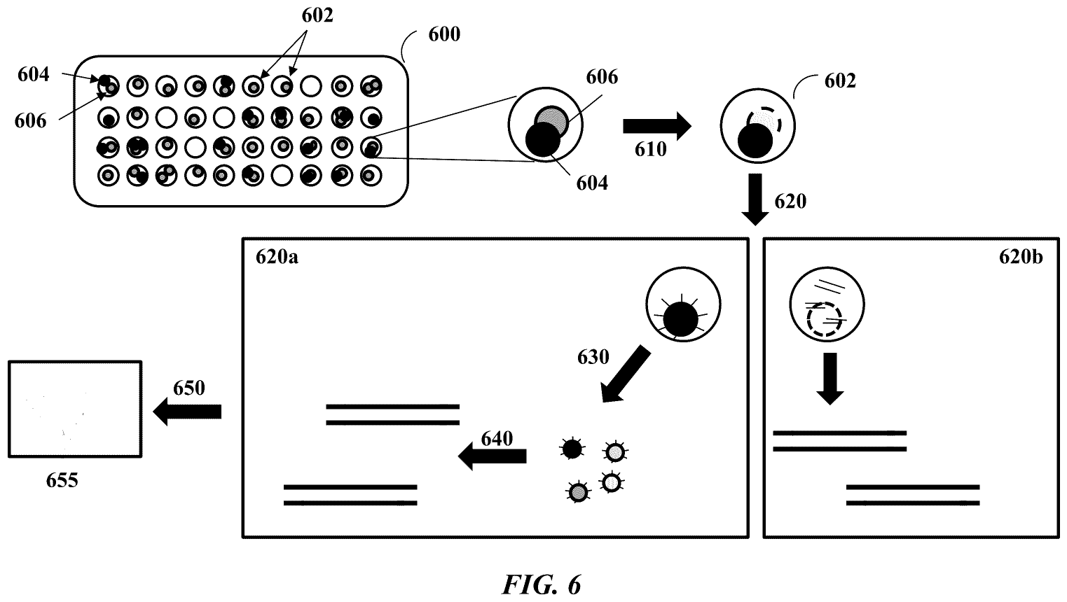

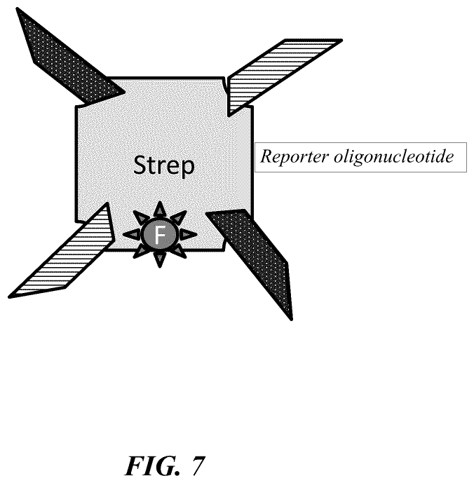

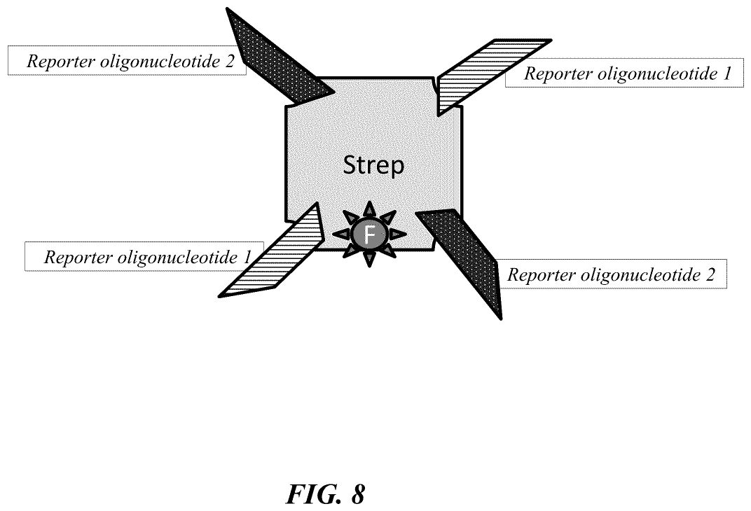

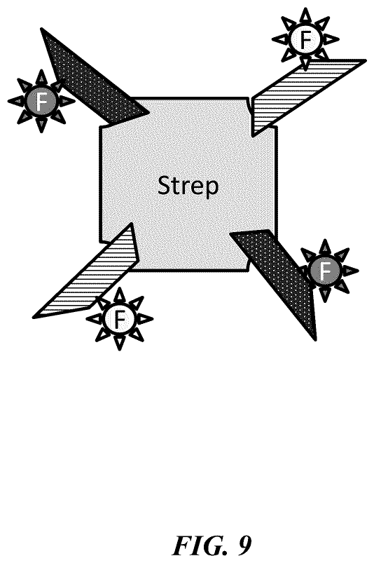

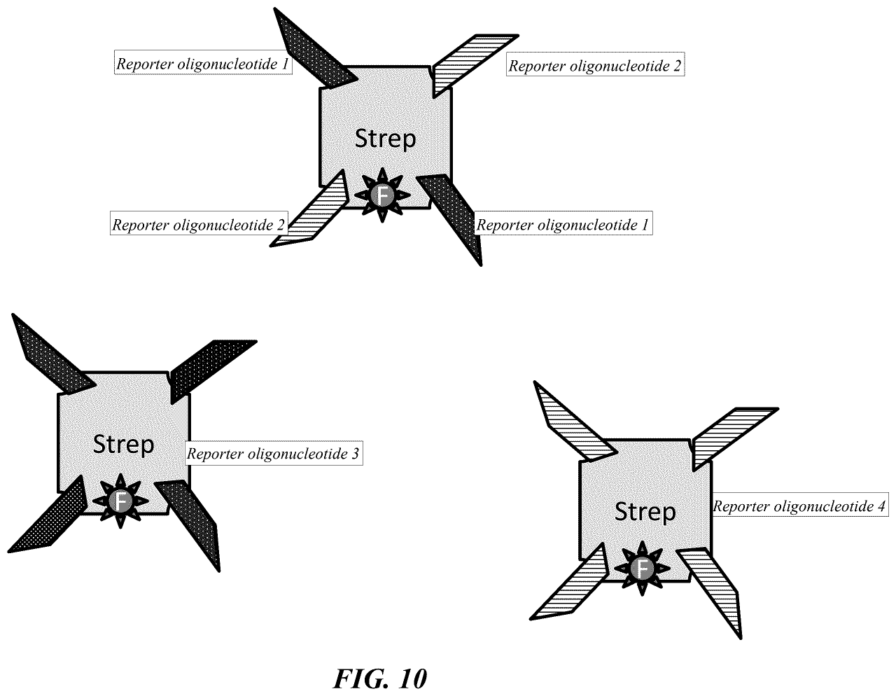

FIG. 6 schematically shows an example workflow for processing nucleic acid molecules within a sample. A substrate 600 comprising a plurality of microwells 602 may be provided. A sample 606 which may comprise a cell, cell bead, cellular components or analytes (e.g., proteins and/or nucleic acid molecules) can be co-partitioned, in a plurality of microwells 602, with a plurality of beads 604 comprising nucleic acid barcode molecules. During process 610, the sample 606 may be processed within the partition. For instance, in the case of live cells, the cell may be subjected to conditions sufficient to lyse the cells and release the analytes contained therein. In process 620, the bead 604 may be further processed. By way of example, processes 620a and 620b schematically illustrate different workflows, depending on the properties of the bead 604.

In 620a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 630, the beads 604 from multiple wells 602 may be collected and pooled. Further processing may be performed in process 640. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

In 620b, the bead comprises nucleic acid barcode molecules that are releasably attached thereto, as described below. The bead may degrade or otherwise release the nucleic acid barcode molecules into the well 602; the nucleic acid barcode molecules may then be used to barcode nucleic acid molecules within the well 602. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

### Sample and Cell Processing

A sample may derive from any useful source including any subject, such as a human subject. A sample may comprise material (e.g., one or more biological particles) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, may be obtained for analysis as described herein. For example, a first sample may be obtained from a subject at a first time and a second sample may be obtained from the subject at a second time later than the first time. The first time may be before a subject undergoes a treatment regimen or procedure (e.g., to address a disease or condition), and the second time may be during or after the subject undergoes the treatment regimen or procedure. In another example, a first sample may be obtained from a first bodily location or system of a subject (e.g., using a first collection technique) and a second sample may be obtained from a second bodily location or system of the subject (e.g., using a second collection technique), which second bodily location or system may be different than the first bodily location or system. In another example, multiple samples may be obtained from a subject at a same time from the same or different bodily locations. Different samples, such as different subjects collected from different bodily locations of a same subject, at different times, from multiple different subjects, and/or using different collection techniques, may undergo the same or different processing (e.g., as described herein). For example, a first sample may undergo a first processing protocol and a second sample may undergo a second processing protocol. In another example, a portion of a sample may undergo a first processing protocol and a second portion of the sample may undergo a second processing protocol.

A sample may be a biological sample, such as a cell sample (e.g., as described herein). A sample may include one or more biological particles, such as one or more cells and/or cellular constituents, such as one or more cell nuclei. A sample may be a tissue sample. For example, a sample may comprise a plurality of biological particles, such as a plurality of cells and/or cellular constituents. Biological particles (e.g., cells or cellular constituents, such as cell nuclei) of a sample may be of a single type or a plurality of different types. For example, cells of a sample may include one or more different types or blood cells.

Cells and cellular constituents of a sample may be of any type. For example, a cell or cellular constituent may be a vertebral, mammalian, fungal, plant, bacterial, or other cell type. In some cases, the cell is a mammalian cell, such as a human cell. The cell may be, for example, a stem cell, liver cell, nerve cell, bone cell, blood cell, reproductive cell, skin cell, skeletal muscle cell, cardiac muscle cell, smooth muscle cell, hair cell, hormone-secreting cell, or glandular cell. The cell may be, for example, an erythrocyte (e.g., red blood cell), a megakaryocyte (e.g., platelet precursor), a monocyte (e.g., white blood cell), a leukocyte, a B cell, a T cell (such as a helper, suppressor, cytotoxic, or natural killer T cell), an osteoclast, a dendritic cell, a connective tissue macrophage, an epidermal Langerhans cell, a microglial cell, a granulocyte, a hybridoma cell, a mast cell, a natural killer cell, a reticulocyte, a hematopoietic stem cell, a myoepithelial cell, a myeloid-derived suppressor cell, a platelet, a thymocyte, a satellite cell, an epithelial cell, an endothelial cell, an epididymal cell, a kidney cell, a liver cell, an adipocyte, a lipocyte, or a neuron cell. In some cases, the cell may be associated with a cancer, tumor, or neoplasm. In some cases, the cell may be associated with a fetus. In some cases, the cell may be a Jurkat cell.

A biological sample may include a plurality of cells having different dimensions and features. In some cases, processing of the biological sample, such as cell separation and sorting (e.g., as described herein), may affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process may comprise the use of microfluidics (e.g., to separate biological particles of different sizes, types, charges, or other features).

In an example, a sample comprising one or more cells may be processed to separate the one or more cells from other materials in the sample (e.g., using centrifugation and/or another process). In some cases, cells and/or cellular constituents of a sample may be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process may comprise a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells). Separation of one or more different types of cells may comprise, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method.

For example, a flow cytometry method may be used to detect cells and/or cellular constituents based on a parameter such as a size, morphology, or protein expression. Flow cytometry-based cell sorting may comprise injecting a sample into a sheath fluid that conveys the cells and/or cellular constituents of the sample into a measurement region one at a time. In the measurement region, a light source such as a laser may interrogate the cells and/or cellular constituents and scattered light and/or fluorescence may be detected and converted into digital signals. A nozzle system (e.g., a vibrating nozzle system) may be used to generate droplets (e.g., aqueous droplets) comprising individual cells and/or cellular constituents. Droplets including cells and/or cellular constituents of interest (e.g., as determined via optical detection) may be labeled with an electric charge (e.g., using an electrical charging ring), which charge may be used to separate such droplets from droplets including other cells and/or cellular constituents. For example, FACS may comprise labeling cells and/or cellular constituents with fluorescent markers (e.g., using internal and/or external biomarkers). Cells and/or cellular constituents may then be measured and identified one by one and sorted based on the emitted fluorescence of the marker or absence thereof. MACS may use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS may use microbubbles (e.g., glass microbubbles) labeled with antibodies to target cells of interest. Cells and/or cellular components coupled to microbubbles may float to a surface of a solution, thereby separating target cells and/or cellular components from other components of a sample. Cell separation techniques may be used to enrich for populations of cells of interest (e.g., prior to partitioning, as described herein). For example, a sample comprising a plurality of cells including a plurality of cells of a given type may be subjected to a positive separation process. The plurality of cells of the given type may be labeled with a fluorescent marker (e.g., based on an expressed cell surface marker or another marker) and subjected to a FACS process to separate these cells from other cells of the plurality of cells. The selected cells may then be subjected to subsequent partition-based analysis (e.g., as described herein) or other downstream analysis. The fluorescent marker may be removed prior to such analysis or may be retained. The fluorescent marker may comprise an identifying feature, such as a nucleic acid barcode sequence and/or unique molecular identifier.

In another example, a first sample comprising a first plurality of cells including a first plurality of cells of a given type (e.g., immune cells expressing a particular marker or combination of markers) and a second sample comprising a second plurality of cells including a second plurality of cells of the given type may be subjected to a positive separation process. The first and second samples may be collected from the same or different subjects, at the same or different types, from the same or different bodily locations or systems, using the same or different collection techniques. For example, the first sample may be from a first subject and the second sample may be from a second subject different than the first subject. The first plurality of cells of the first sample may be provided a first plurality of fluorescent markers configured to label the first plurality of cells of the given type. The second plurality of cells of the second sample may be provided a second plurality of fluorescent markers configured to label the second plurality of cells of the given type. The first plurality of fluorescent markers may include a first identifying feature, such as a first barcode, while the second plurality of fluorescent markers may include a second identifying feature, such as a second barcode, that is different than the first identifying feature. The first plurality of fluorescent markers and the second plurality of fluorescent markers may fluoresce at the same intensities and over the same range of wavelengths upon excitation with a same excitation source (e.g., light source, such as a laser). The first and second samples may then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples may undergo separate FACS processes and the positively selected cells of the given type from the first sample and the positively selected cells of the given type from the second sample may then be combined for subsequent analysis. The encoded identifying features of the different fluorescent markers may be used to identify cells originating from the first sample and cells originating from the second sample. For example, the first and second identifying features may be configured to interact (e.g., in partitions, as described herein) with nucleic acid barcode molecules (e.g., as described herein) to generate barcoded nucleic acid products detectable using, e.g., nucleic acid sequencing.

### Multiplexing

The present disclosures provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize biological particles and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, an MHC molecule complex, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In some aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more biological particles may be used to characterize a biological particle as belonging to a particular set of biological particles. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a biological particle may comprise subjecting the antibody-linked barcode molecule or the epitope binding fragment-linked barcode molecule to conditions suitable for binding the antibody to a molecule present on a surface of the biological particle. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 M, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into a biological particle. Labeling biological particles may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in embodiments herein include penetratin, transportan, plsl, TAT (48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the biological particle. In some instances, fluorophores can interact strongly with lipid bilayers and labeling biological particles may comprise subjecting the fluorophore-linked barcode molecule to conditions such that the fluorophore binds to or is inserted into a membrane of the biological particle. In some cases, the fluorophore is a water-soluble, organic fluorophore. In some instances, the fluorophore is Alexa 532 maleimide, tetramethylrhodamine-5-maleimide (TMR malcimide), BODIPY-TMR malcimide, Sulfo-Cy3 malcimide, Alexa 546 carboxylic acid/succinimidyl ester, Atto 550 malcimide, Cy3 carboxylic acid/succinimidyl ester, Cy3B carboxylic acid/succinimidyl ester, Atto 565 biotin, Sulforhodamine B, Alexa 594 malcimide, Texas Red malcimide, Alexa 633 maleimide, Abberior STAR 635P azide, Atto 647N maleimide, Atto 647 SE, or Sulfo-Cy5 maleimide. Sec, e.g., Hughes L D, et al. PLOS Onc. 2014 Feb. 4; 9 (2): c87649, which is hereby incorporated by reference in its entirety for all purposes, for a description of organic fluorophores.

A reporter oligonucleotide may be coupled to a lipophilic molecule, and labeling biological particles may comprise delivering the nucleic acid barcode molecule to a membrane of the biological particle or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and biological particle may be such that the biological particle retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular biological particle, population, or sample. The biological particles may be incubated with a plurality of libraries and a given biological particle may comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment), e.g., via a linker, using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. Sec, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. Sec, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

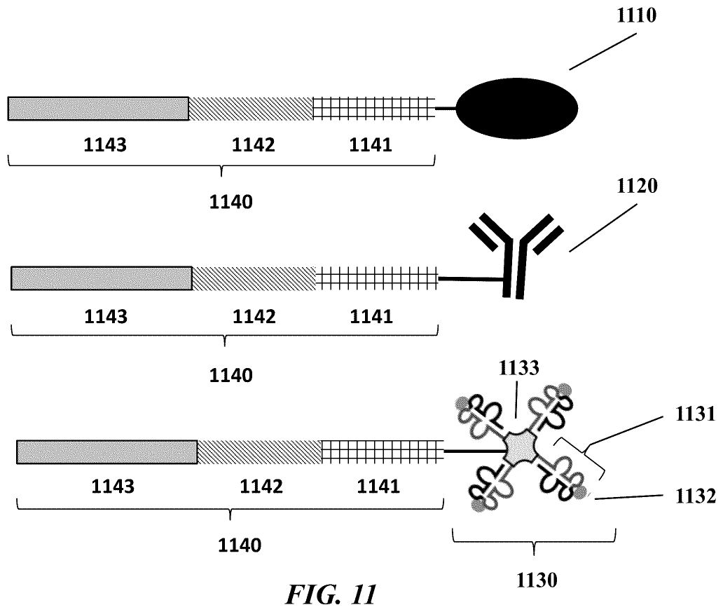

FIG. 11 describes exemplary labelling agents (1110, 1120, 1130) comprising reporter oligonucleotides (1140) attached thereto. Labelling agent 1110 (e.g., any of the labelling agents described herein) is attached (either directly, e.g., covalently attached, or indirectly) to reporter oligonucleotide 1140. Reporter oligonucleotide 1140 may comprise barcode sequence 1142 that identifies labelling agent 1110. Reporter oligonucleotide 1140 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 11, in some instances, reporter oligonucleotide 1140 conjugated to a labelling agent (e.g., 1110, 1120, 1130) comprises a functional sequence 1141 (e.g., a primer sequence), a barcode sequence that identifies the labelling agent (e.g., 1110, 1120, 1130), and functional sequence 1143. Functional sequence 1143 can be a reporter capture handle sequence configured to hybridize to a complementary sequence, such as a complementary sequence present on a nucleic acid barcode molecule 1190 (not shown), such as those described elsewhere herein. In some instances, nucleic acid barcode molecule 1190 is attached to a support (e.g., a bead, such as a gel bead), such as those described elsewhere herein. For example, nucleic acid barcode molecule 1190 may be attached to the support via a releasable linkage (e.g., comprising a labile bond), such as those described elsewhere herein. In some instances, reporter oligonucleotide 1140 comprises one or more additional functional sequences, such as those described above.

In some instances, the labelling agent 1110 is a protein or polypeptide (e.g., an MHC molecule complex, an antigen or prospective antigen) comprising reporter oligonucleotide 1140. Reporter oligonucleotide 1140 comprises barcode sequence 1142 that identifies polypeptide 1110 and can be used to infer the presence of an analyte, e.g., a binding partner of polypeptide 1110 (i.e., a molecule or compound to which polypeptide 1110 can bind). In some instances, the labelling agent 1110 is a lipophilic moiety (e.g., cholesterol) comprising reporter oligonucleotide 1140, where the lipophilic moiety is selected such that labelling agent 1110 integrates into a membrane of a cell or nucleus. Reporter oligonucleotide 1140 comprises barcode sequence 1142 that identifies lipophilic moiety 1110 which in some instances is used to tag cells (e.g., groups of cells, cell samples, etc.) and may be used for multiplex analyses as described elsewhere herein. In some instances, the labelling agent is an antibody 1120 (or an epitope binding fragment thereof) comprising reporter oligonucleotide 1140. Reporter oligonucleotide 1140 comprises barcode sequence 1142 that identifies antibody 1120 and can be used to infer the presence of, e.g., a target of antibody 1120 (i.e., a molecule or compound to which antibody 1120 binds). In other embodiments, labelling agent 1130 comprises an MHC molecule 1131 comprising peptide 1132 and reporter oligonucleotide 1140 that identifies peptide 1132. In some instances, the MHC molecule is coupled to a support 1133. In some instances, support 1133 may be or comprise a polypeptide, such as avidin, neutravidin, streptavidin, or a polysaccharide, such as dextran. In some embodiments, support 1133 further comprises a detectable label, e.g., a detectable label described herein, e.g., a fluorescent label. In some instances, reporter oligonucleotide 1140 may be directly or indirectly coupled to MHC labelling agent 1130 in any suitable manner. For example, reporter oligonucleotide 1140 may be coupled to MHC molecule 1131, support 1133, or peptide 1132. In some embodiments, labelling agent 1130 comprises a plurality of MHC molecules described herein, (e.g. is an MHC multimer, which may be coupled to a support (e.g., 1133)). In some embodiments, reporter oligonucleotide 1140 and MHC molecule 1130 are attached to the polypeptide or polysaccharide of support 1133. In some embodiments, reporter oligonucleotide 1140 and MHC molecule 1130 are attached to the detectable label of support 1133. In some embodiments, reporter oligonucleotide 1140 and an antigen (e.g., protein, polypeptide) are attached to polypeptide or polysaccharide of support 1133. In some embodiments, reporter oligonucleotide 1140 and an antigen (e.g., protein, polypeptide) are attached to the detectable label of support 1133. There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

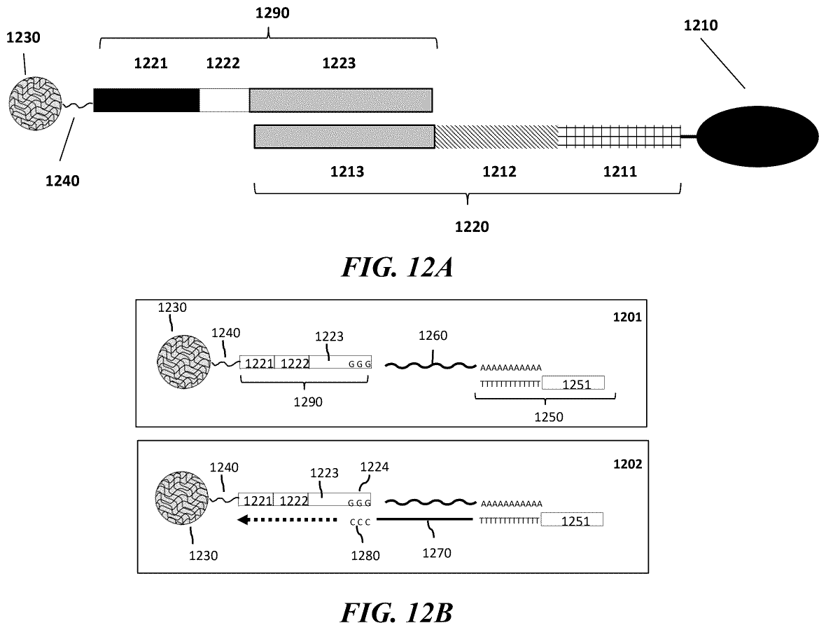

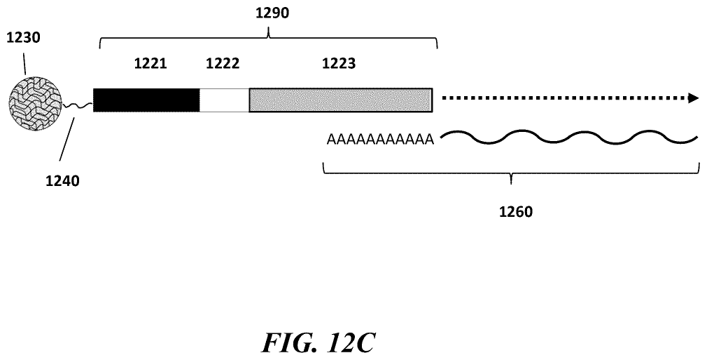

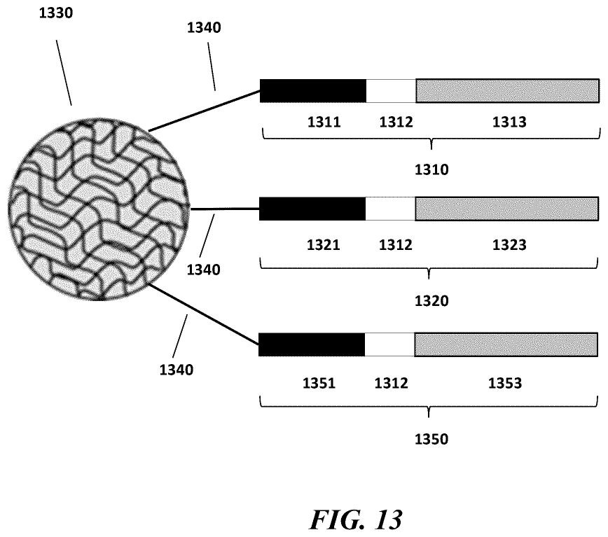

FIG. 13 illustrates another example of a barcode carrying bead. In some embodiments, analysis of multiple analytes (e.g., RNA and one or more analytes using labelling agents described herein) may comprise nucleic acid barcode molecules as generally depicted in FIG. 13. In some embodiments, nucleic acid barcode molecules 1310 and 1320 are attached to support 1330 via a releasable linkage 1340 (e.g., comprising a labile bond) as described elsewhere herein. Nucleic acid barcode molecule 1310 may comprise adapter sequence 1311, barcode sequence 1312 and capture sequence 1313. Nucleic acid barcode molecule 1320 may comprise adapter sequence 1321, barcode sequence 1312, and capture sequence 1323, wherein capture sequence 1323 comprises a different sequence than capture sequence 1313. In some instances, adapter 1311 and adapter 1321 comprise the same sequence. In some instances, adapter 1311 and adapter 1321 comprise different sequences. Although support 1330 is shown comprising nucleic acid barcode molecules 1310 and 1320, any suitable number of barcode molecules comprising common barcode sequence 1312 are contemplated herein. For example, in some embodiments, support 1330 further comprises nucleic acid barcode molecule 1350. Nucleic acid barcode molecule 1350 may comprise adapter sequence 1351, barcode sequence 1312 and capture sequence 1353, wherein capture sequence 1353 comprises a different sequence than capture sequence 1313 and 1323. In some instances, nucleic acid barcode molecules (e.g., 1310, 1320, 1350) comprise one or more additional functional sequences, such as a UMI or other sequences described herein. The nucleic acid barcode molecules 1310, 1320 or 1350 may interact with analytes as described elsewhere herein, for example, as depicted in FIGS. 12A-C.

Referring to FIG. 12A, in an instance where cells are labelled with labeling agents, capture sequence 1223 may be complementary to an adapter sequence of a reporter oligonucleotide. Cells may be contacted with one or more reporter oligonucleotide 1220 conjugated labelling agents 1210 (e.g., MHC molecule complex, polypeptide, antibody, or others described elsewhere herein). In some cases, the cells may be further processed prior to barcoding. For example, such processing steps may include one or more washing and/or cell sorting steps. In some instances, a cell that is bound to labelling agent 1210 which is conjugated to oligonucleotide 1220 and support 1230 (e.g., a bead, such as a gel bead) comprising nucleic acid barcode molecule 1290 is partitioned into a partition amongst a plurality of partitions (e.g., a droplet of a droplet emulsion or a well of a microwell array). In some instances, the partition comprises at most a single cell bound to labelling agent 1210. In some instances, reporter oligonucleotide 1220 conjugated to labelling agent 1210 (e.g., polypeptide, an antibody, pMHC molecule such as an MHC multimer, etc.) comprises a first adapter sequence 1211 (e.g., a primer sequence), a barcode sequence 1212 that identifies the labelling agent 1210 (e.g., the polypeptide, antibody, or peptide of a pMHC molecule or complex), and an capture handle sequence 1213. Capture handle sequence 1213 may be configured to hybridize to a complementary sequence, such as a capture sequence 1223 present on a nucleic acid barcode molecule 1290. In some instances, oligonucleotide 1220 comprises one or more additional functional sequences, such as those described elsewhere herein.

Barcoded nucleic may be generated (e.g., via a nucleic acid reaction, such as nucleic acid extension or ligation) from the constructs described in FIGS. 12A-C. For example, capture handle sequence 1213 may then be hybridized to complementary sequence, such as capture sequence 1223 to generate (e.g., via a nucleic acid reaction, such as nucleic acid extension or ligation) a barcoded nucleic acid molecule comprising cell (e.g., partition specific) barcode sequence 1222 (or a reverse complement thereof) and reporter barcode sequence 1212 (or a reverse complement thereof). In some embodiments, the nucleic acid barcode molecule 1290 (e.g., partition-specific barcode molecule) further includes a UMI (not shown). Barcoded nucleic acid molecules can then be optionally processed as described elsewhere herein, e.g., to amplify the molecules and/or append sequencing platform specific sequences to the fragments. Sec, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 12A-C, or a combination of workflows for an individual analyte, as described elsewhere herein. For example, by using a combination of the workflows as generally depicted in FIGS. 12A-C, multiple analytes can be analyzed.

In some instances, analysis of an analyte (e.g. a nucleic acid, a polypeptide, a carbohydrate, a lipid, TCR or TCR-like ABM etc.) comprises a workflow as generally depicted in FIG. 12A. A nucleic acid barcode molecule 1290 may be co-partitioned with the one or more analytes. In some instances, nucleic acid barcode molecule 1290 is attached to a support 1230 (e.g., a bead, such as a gel bead), such as those described elsewhere herein. For example, nucleic acid barcode molecule 1290 may be attached to support 1230 via a releasable linkage 1240 (e.g., comprising a labile bond), such as those described elsewhere herein. Nucleic acid barcode molecule 1290 may comprise a functional sequence 1221 and optionally comprise other additional sequences, for example, a barcode sequence 1222 (e.g., common barcode, partition-specific barcode, or other functional sequences described elsewhere herein), and/or a UMI sequence (not shown) . . . . The nucleic acid barcode molecule 1290 may comprise a capture sequence 1223 that may be complementary to another nucleic acid sequence, such that it may hybridize to a particular sequence, e.g., capture handle sequence 1213.

For example, capture sequence 1223 may comprise a poly-T sequence and may be used to hybridize to mRNA. Referring to FIG. 12C, in some embodiments, nucleic acid barcode molecule 1290 comprises capture sequence 1223 complementary to a sequence of RNA molecule 1260 from a cell. In some instances, capture sequence 1223 comprises a sequence specific for an RNA molecule. Capture sequence 1223 may comprise a known or targeted sequence or a random sequence. In some instances, a nucleic acid extension reaction may be performed, thereby generating a barcoded nucleic acid product comprising capture sequence 1223, the functional sequence 1221, barcode sequence 1222, any other functional sequence, and a sequence corresponding to the RNA molecule 1260.

In another example, capture sequence 1223 may be complementary to an overhang sequence or an adapter sequence that has been appended to an analyte. For example, referring to FIG. 12B, panel 1201, in some embodiments, primer 1250 comprises a sequence complementary to a sequence of nucleic acid molecule 1260 (such as an RNA encoding for a TCR or BCR sequence) from a biological particle. In some instances, primer 1250 comprises one or more sequences 1251 that are not complementary to RNA molecule 1260. Sequence 1251 may be a functional sequence as described elsewhere herein, for example, an adapter sequence, a sequencing primer sequence, or a sequence the facilitates coupling to a flow cell of a sequencer. In some instances, primer 1250 comprises a poly-T sequence. In some instances, primer 1250 comprises a sequence complementary to a target sequence in an RNA molecule. In some instances, primer 1250 comprises a sequence complementary to a region of an immune molecule, such as the constant region of a TCR or BCR sequence. Primer 1250 is hybridized to nucleic acid molecule 1260 and complementary molecule 1270 is generated (see Panel 1202). For example, complementary molecule 1270 may be cDNA generated in a reverse transcription reaction. In some instances, an additional sequence may be appended to complementary molecule 1270. For example, the reverse transcriptase enzyme may be selected such that several non-templated bases 1280 (e.g., a poly-C sequence) are appended to the cDNA. In another example, a terminal transferase may also be used to append the additional sequence. Nucleic acid barcode molecule 1290 comprises a sequence 1224 complementary to the non-templated bases, and the reverse transcriptase performs a template switching reaction onto nucleic acid barcode molecule 1290 to generate a barcoded nucleic acid molecule comprising cell (e.g., partition specific) barcode sequence 1222 (or a reverse complement thereof) and a sequence of complementary molecule 1270 (or a portion thereof). In some instances, sequence 1223 comprises a sequence complementary to a region of an immune molecule, such as the constant region of a TCR or BCR sequence. Sequence 1223 is hybridized to nucleic acid molecule 1260 and a complementary molecule 1270 is generated. For example complementary molecule 1270 may be generated in a reverse transcription reaction generating a barcoded nucleic acid molecule comprising cell (e.g., partition specific) barcode sequence 1222 (or a reverse complement thereof) and a sequence of complementary molecule 1270 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. Sec, e.g., Xu J. et al., Genome Biology Vol. 20, 290 (2019); Huang Y. et al., Genome Biology Vol. 20, 273 (2019); and Heaton et al., Nature Methods volume 17, pages 615-620 (2020).

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (BCR), and other highly polymorphic regions of the genome), can also be used for demultiplexing. Expressed B cell receptors can be used to infer germline alleles from unrelated individuals, which information may be used for demultiplexing.

### Combinatorial Barcoding

In some instances, barcoding of a nucleic acid molecule may be done using a combinatorial approach. In such instances, one or more nucleic acid molecules (which may be comprised in a biological particle, e.g., a cell, e.g., a fixed cell, organelle, nucleus, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The barcoded nucleic acid molecules may thus comprise two barcode sequences (e.g., from the first nucleic acid barcode molecules and the second nucleic acid barcode molecules).

Additional barcode sequences may be attached to the barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a barcoded nucleic acid molecule. A second unique nucleic acid molecule comprising one or more nucleic acid bases may be attached to the barcoded nucleic acid molecule molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

### Alternative Partitioning Methods

In some instances wherein a partition is a volume wherein diffusion of contents beyond the volume is inhibited, the partition contains a diffusion resistant material. Such partition may also be referred to herein as a diffusion resistant partition. The diffusion resistant material may have an increased viscosity. The diffusion resistant material may be or comprise a matrix, e.g., a polymeric matrix, or a gel. Suitable polymers or gels are disclosed herein. The matrix can be a porous matrix capable of entraining and/or retaining materials within its matrix. In some embodiments, a diffusion resistant partition comprises a single biological particle and a single bead, the single bead comprising a plurality of nucleic acid barcode molecules comprising a partition specific barcode sequence. In some embodiments the partition specific barcode sequence is unique to the diffusion resistant partition. In some embodiments, partitioning comprises contacting a plurality of biological particles with a plurality of beads in a diffusion resistant material to provide a diffusion resistant partition comprising a single biological particle and a single bead. In some embodiments, partitioning comprises contacting a plurality of biological particles with a plurality of beads in a liquid comprising a polymeric precursor material that may be capable of being formed into a gel or other solid or semi-solid matrix, and subjecting the liquid to conditions sufficient to polymerize or gel the precursors, e.g., as described herein. In some embodiments, the biological particle may be lysed or permeabilized in the diffusion resistant partition. In some embodiments, a nucleic acid analyte of the biological particle (which may include a reporter oligonucleotide associated with a labelling agent disclosed herein) may be coupled with a nucleic acid barcode molecule in the diffusion resistant partition. In some cases, further processing, e.g., generation of barcoded nucleic acid molecules, may be performed in the diffusion resistant partition or in bulk. For example, nucleic acid analytes, once coupled to nucleic acid barcode molecules in partitions, may be pooled and then subjected to further processing in bulk (e.g, extension, reverse transcription, or other processing) to generate barcoded nucleic acid molecules. For other example, nucleic acid analytes, one coupled to nucleic acid barcode molecules in diffusion resistant partitions, may be subjected to further processing in the diffusion resistant partitions to generate barcoded nucleic acid molecules.

### Computer Systems

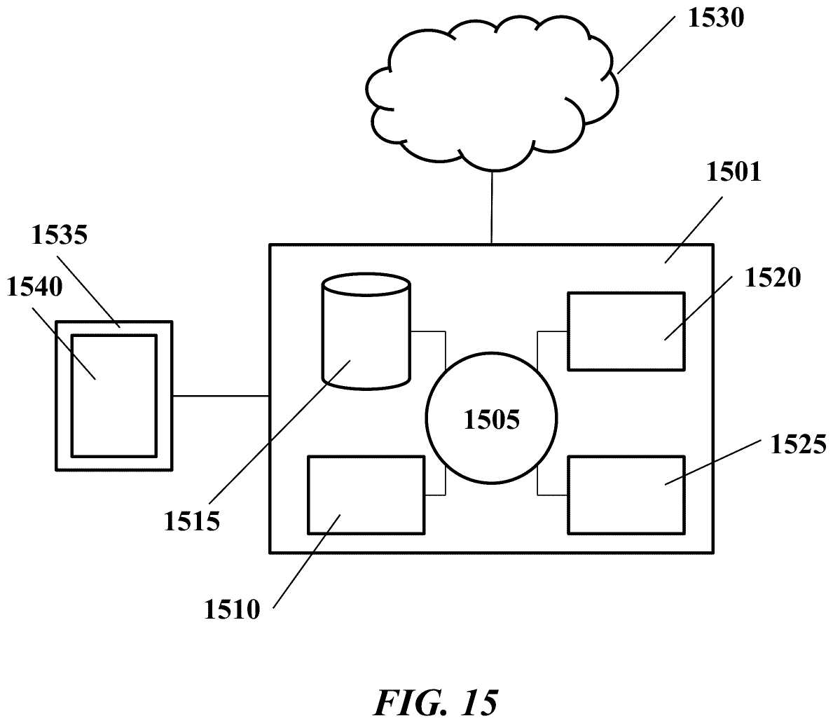

The present disclosure provides computer systems that are programmed to implement methods of the disclosure. FIG. 15 shows a computer system 1501 that is programmed or otherwise configured to: (i) control a microfluidics system (e.g., fluid flow), (ii) detect fluorescent signals, (iii) perform sequencing applications, and/or (iv) generate and maintain a library of sequences from barcoded nucleic acid molecules. The computer system 1501 can regulate various aspects of the present disclosure, such as, for example, e.g., regulating fluid flow rate in one or more channels in a microfluidic structure. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1420 (e.g., network adapter) for communicating with one or more other systems, and peripheral devices 1525, such as cache, other memory, data storage and/or electronic display adapters. The memory 14510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, results of sequencing analysis, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, e.g., perform sequencing, etc.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Additional embodiments are disclosed in further detail in the following examples, which are provided by way of illustration and are not in any way intended to limit the scope of this disclosure or the claims.

### EXAMPLES

### Example 1

### Target Reagent Preparation

First and second target antigens, e.g., human metapneumovirus (HMPV) pre-fusion glycoprotein and Respiratory Syncytial virus (RSV) pre-glycoprotein, are biotinylated and conjugated to core supports, e.g., streptavidin complexes of streptavidin molecules to form first target reagents. To biotinylate the first and second target antigens, e.g., HMPV and RSV pre-fusion glycoproteins, a biotin (Sigma, Cat. #B4639-500 MG, lot #SLCB9976) and PBS (Ca2+Mg2+ free) solution is prepared. HMPV and RSV pre-fusion glycoproteins are separately biotinylated using the biotin and PBS solution. Biotinylated HMPV and RSV pre-fusion glycoproteins are combined in a 1:1 ratio and are added to the core support at a 5:1 ratio of biotinylated HMPV/RSV pre-fusion glycoprotein to core support. In some embodiments, the core support includes a reporter oligonucleotide. In other embodiments, the first and the second, e.g., HMPV and RSV pre-fusion glycoproteins, respectively, are coupled to first and second reporter oligonucleotides, respectively. In some embodiments, the core support includes a fluorescent molecule. In other embodiments, the the first and the second, e.g., HMPV and RSV pre-fusion glycoproteins, are coupled to first and second fluorescent molecules, respectively. In addition to preparing the target reagent including the first and the second target antigens, HMPV and RSV pre-fusion glycoproteins, second and third target reagents may be prepared, e.g., a second target reagent to which the HMPV pre-fusion glycoprotein is conjugated to a core support and a third target reagent to which the RSV pre-fusion glycoprotein is conjugated to a core support.

### Example 2

Incubating Cells with Target Reagents

A plurality of cells having ABMs is incubated with the target reagent, e.g., including the HMPV and the RSV pre-fusion glycoproteins, (or the target reagent, second target reagent, and third target reagent) in the dark for 30 minutes on ice. The cells are washed 3 times with 2 mL of cold labeling buffer at 350*g for 5 minutes at 4° C., then resuspended in cold labeling buffer and a 1:200 addition of live/dead cell discriminating agent 7AAD for 10 minutes on ice in the dark. The cells are then washed one more time with labeling buffer at 350*g for 5 minutes at 4° C., then resuspended in labeling buffer and loaded into a Sony MA900 Cell Sorter using a 70 microM sorting chip.

### Example 3

### Analysis

The cells are analyzed by FACS analysis. To prepare for FACS analysis, the cells are washed with FBS in PBS and resuspended in FBS in PBS. Cells are initially gated on being single, live (7AAD^{negative}) and then may be sorted based on having bound to the target reagent in instances in which the target reagent includes a fluorescent molecule, e.g., fluorescent molecule conjugated to the core support, the first target antigen or second target antigen. FACS data are analyzed using FlowJo. Standard gene expression, V(D)J, and barcoded antigen libraries are constructed using the 10×5′V2 Single Cell Immune Profiling kit per manufacturer's instructions. Additional information in this regard can be found at “support. 10×genomics.com/permalink/getting-started-immune-profiling-feature-barcoding.”

While particular alternatives of the present disclosure have been disclosed, it is to be understood that various modifications and combinations are possible and arc contemplated within the true spirit and scope of the appended claims. There is no intention, therefore, of limitations to the exact abstract and disclosure herein presented.

1. A method for characterizing an antigen-binding molecule (ABM), the method comprising:
(a) contacting a plurality of cells expressing ABMs with a target reagent comprising:
(i) a first target antigen,
(ii) a second target antigen, and
(iii) a first reporter oligonucleotide;
(b) partitioning the plurality of cells expressing ABMs and the target reagent into a plurality of partitions, wherein a partition comprises:
(i) a cell expressing an ABM bound to the target reagent, and
(ii) a plurality of nucleic acid barcode molecules comprising a partition-specific barcode sequence;
(c) generating barcoded nucleic acid molecules, wherein the barcoded nucleic acid molecules comprise:
(i) a first barcoded nucleic acid molecule comprising a nucleic acid sequence encoding at least a portion of the ABM expressed by the cell or reverse complement thereof and the partition-specific barcode sequence or reverse complement thereof, and
(ii) a second barcoded nucleic acid molecule comprising a sequence of the first reporter oligonucleotide or a reverse complement thereof and the partition-specific barcode sequence or reverse complement thereof,
wherein generation of the second barcoded nucleic acid molecule characterizes the ABM as having specificity for the first and the second target antigen.

2. The method of claim 1, wherein the first target reagent further comprises:
(d) an additional reporter oligonucleotide,
wherein the first target antigen is coupled to the first reporter oligonucleotide and the second target antigen is coupled to the additional reporter oligonucleotide.

3. The method of claim 2, wherein the barcoded nucleic acid molecules generated at (c) further comprise an additional barcoded nucleic acid molecule, wherein the additional barcoded nucleic acid molecule comprises a sequence of the additional reporter oligonucleotide or a reverse complement thereof and the partition-specific barcode sequence or reverse complement thereof.

4. The method of claim 1, further comprising in (a) contacting the plurality of cells expressing ABMs with a second target reagent and a third target reagent,
wherein the second target reagent comprises the first target antigen and a second reporter oligonucleotide; and
wherein the third target reagent comprises the second target antigen and a third reporter oligonucleotide.

5. The method of claim 4, wherein the cell that had bound to the first target reagent further had bound to:
(i) the second and the third target reagent,
wherein the barcoded nucleic acid molecules generated at (c) further comprise third and fourth barcoded nucleic acid molecules,
wherein the third barcoded nucleic acid molecule comprises a sequence of the second reporter oligonucleotide or a reverse complement thereof and the partition-specific barcode sequence or reverse complement thereof,
wherein the fourth barcoded nucleic molecule comprises a sequence of the third reporter oligonucleotide or reverse complement thereof and the partition-specific barcode sequence or reverse complement thereof;
(ii) the second, but not the third, target reagent,
wherein the barcoded nucleic acid molecules generated at (c) further comprise a third barcoded nucleic acid molecule,
wherein the third barcoded nucleic acid molecule comprises a sequence of the second reporter oligonucleotide or a reverse complement thereof and the partition-specific barcode sequence or reverse complement thereof; or
(iii) the third, but not the second, target reagent,
wherein the barcoded nucleic acid molecules generated at (c) further comprise a third barcoded nucleic acid molecule,
wherein the third barcoded nucleic acid molecule comprises a sequence of the third reporter oligonucleotide or a reverse complement thereof and the partition-specific barcode sequence or reverse complement thereof.

6. The method of claim 5, further comprising characterizing the candidate ABM as having specificity for:
(I) both the first and the second target antigen based on the generated third and fourth barcoded nucleic acid molecules at (i);
(II) the first target antigen based on the generated third barcoded nucleic acid molecule at (ii); or
(III) the second target antigen based on the generated third barcoded nucleic acid molecule at (iii).

7. The method of claim 1, wherein the first target reagent further comprises a first detectable label.

8. The method of claim 7, wherein, prior to the (b) partitioning, sorting for cells of the plurality of cells comprising the detectable label.

9. The method of claim 1, wherein:
(i) a first nucleic acid barcode molecule of the plurality of nucleic acid barcode molecules further comprises a capture sequence configured to couple to (a) an mRNA or DNA analyte or (b) non-templated nucleotides appended to a cDNA reverse transcribed from an mRNA analyte; and
(ii) a second nucleic acid barcode molecule of the plurality of nucleic acid barcode molecules further comprises a capture sequence configured to couple to a capture handle sequence further comprised in the first reporter oligonucleotide.

10. A method for characterizing an ABM, the method comprising:
(a) contacting a plurality of cells expressing ABMs with a target reagent comprising:
(i) a first target antigen,
(ii) a second target antigen, and
(iii) a first fluorescent molecule;
(b) partitioning the plurality of cells expressing ABMs and the target reagent into a plurality of partitions, wherein a partition comprises:
(i) a cell expressing an ABM bound to the first target reagent, and
(ii) a plurality of nucleic acid barcode molecules comprising a partition-specific barcode sequence; and
(c) generating a first barcoded nucleic acid molecule comprising a nucleic acid sequence encoding at least a portion of the ABM expressed by the cell or reverse complement thereof and the partition-specific barcode sequence or reverse complement thereof.

11. The method of claim 10, further comprising characterizing the ABM based on the generated first barcoded nucleic acid molecule.

12. The method of claim 10, further comprising, prior to the (b) partitioning, sorting for cells of the plurality of cells that comprise the first fluorescent molecule.

13. The method of claim 10, wherein the first target reagent further comprises an additional fluorescent molecule,
wherein the first target antigen is coupled to the first fluorescent molecule and the second target antigen is coupled to the additional fluorescent molecule,
wherein the first fluorescent molecule emits a first detectable signal and the additional fluorescent molecule emits an additional detectable signal.

14. The method of claim 13, further comprising, prior to the (b) partitioning, sorting for cells of the plurality of cells that comprise the first and the additional fluorescent molecule.

15. The method of any of claim 10, further comprising:
a second target reagent and a third target reagent,
wherein the second target reagent comprises the first target antigen and a second fluorescent molecule; and
wherein the third target reagent comprises the second target antigen and a third fluorescent molecule.

16. The method of claim 15, further comprising, prior to the (a) partitioning, sorting for cells of the plurality of cells that comprise the first, the second and the third fluorescent molecule.

17. The method of claim 10, wherein a first nucleic acid barcode molecule of the plurality of barcode molecules further comprises a capture sequence configured to couple to: (a) an mRNA or DNA analyte or (b) non-templated nucleotides appended to a cDNA reverse transcribed from an mRNA analyte.

18. The method of claim 1, further comprising determining the nucleic acid sequence encoding at least the portion of the ABM from the first barcoded nucleic acid molecule.

19. The method of claim 1, wherein the reaction mixture further comprises a control reagent.

20. The method of claim 19, wherein the control reagent comprises a control antigen and a control reporter oligonucleotide.


SEQUENCE LISTING (10 sequences)

SEQ ID NO: 1 - protein, 9 aa, synthetic construct
ALAAAAAAV

SEQ ID NO: 2 - protein, 9 aa, synthetic construct
ATAAAAAAK

SEQ ID NO: 3 - protein, 9 aa, synthetic construct
AYAAAAAAL

SEQ ID NO: 4 - protein, 9 aa, synthetic construct
APAAAAAAV

SEQ ID NO: 5 - protein, 9 aa, synthetic construct
RYAAAAALL

SEQ ID NO: 6 - protein, 10 aa, synthetic construct
TPGPGVRYPL

SEQ ID NO: 7 - protein, 9 aa, synthetic construct
SLYNTVATL

SEQ ID NO: 8 - protein, 10 aa, synthetic construct
TPGPGVRYPL

SEQ ID NO: 9 - protein, 8 aa, synthetic construct
ASYAAAAV

SEQ ID NO: 10 - protein, 8 aa, synthetic construct
TSYKFESV